(19) United States
(12) Patent Application Publication
(10) Pub. No.: US 2023/0070758 A1
(43) Pub. Date: Mar. 9, 2023

(54) Methods of Treating Alzheimer's Disease

(71) Applicant: Athira Pharma, Inc., Bothell, WA (US)

(72) Inventors: Hans J. Moebius, Wollerau (CH); Xue Hua, Bothell, WA (US); Kevin Church, Mountlake Terrace, WA (US); William Walker, Kirkland, WA (US); Leen Kawas, Lynnwood, WA (US)

(73) Assignee: Athira Pharma, Inc., Bothell, WA (US)

(21) Appl. No.: 17/864,702

(22) Filed: Jul. 14, 2022

(30) Foreign Application Priority Data
Jul. 16, 2021 (US) PCT/US2021/042071
Jun. 21, 2022 (US) PCT/US2022/034386

(51) Int. Cl. A61K 38/05; A61K 9/00; A61P 25/28

(52) U.S. Cl. CPC A61K 38/05; A61K 9/0019; A61P 25/28

(57) ABSTRACT

The present disclosure relates to a method of treating mild to moderate Alzheimer's disease comprising administering to a subject with Alzheimer's disease ATH-1017.

### CROSS-REFERENCE TO RELATED APPLICATIONS

This application claims the benefit of priority under 35 U.S.C. §§ 119 and 365 of International Application No. PCT/US2021/042071, filed Jul. 16, 2021, and International Application No. PCT/US2022/034386, filed Jun. 21, 2022, each of which is incorporated by reference herein in its entirety for any purpose.

### FIELD

The present disclosure relates to methods of treating mild to moderate Alzheimer's disease.

### BACKGROUND

Dementia of the Alzheimer's type (hereafter referred to as AD) is the most common form of dementia and the largest unmet medical need in neurology. AD can be further classified based on age of onset and genetic risks. Individuals under age 65 have early-onset AD, and many of whom have a dominant genetic mutation (i.e., familial AD with known mutations in the following genes: amyloid precursor protein, presenilin-1, and presenilin-2). Late-onset AD patients have an age of onset at 65 years and older, who typically have no dominant genetic risks (i.e., sporadic AD), with disease onset involving a complex interplay of aging, Apolipoprotein E (ApoE)-ε4 genotype, environmental, and lifestyle risk factors. The late-onset sporadic cases account for about 95% of the total AD population. Although age is the biggest risk factor, AD is not a part of normal aging.

Growing evidence suggests that complex CNS disorders, like AD, are unlikely to be caused by a single route of pathology; they are likely the result of a multifactorial interplay related to genetics, age, and environment. Pharmacological stimulation of a critical neurotrophic factor system (hepatocyte growth factor, HGF/MET) may stop neurodegeneration and promote neuro-regeneration. Neurotrophic factor system represents a promising therapeutic target for the treatment of AD and other dementias, and drugs that stimulate these systems have the potential to address neurodegeneration and improve cognition by protecting existing neurons, promoting connectivity, inducing neuro-regenerative mechanisms, as well as addressing multiple aspects of the AD pathology, by decreasing inflammation and improving cerebral blood flow (Funakoshi, 2011). The therapeutic promise of neurotrophic factors in neurodegenerative disorders is hampered by the lack of efficient and non-invasive delivery to the brain. Gene therapy strategies, primarily using adeno-associated viral vectors, have been developed and clinically evaluated for therapeutic potential in AD and Parkinson's disease patients. These strategies are largely hindered by challenges related to gene delivery and transduction with limited brain exposure, uncontrollable dose over long-term treatment, and potential immune complications (Piguet, 2017).

Therefore, a small molecule approach capable of passing the blood brain barrier and entering all regions of the brain, presents a superior therapeutic strategy for targeting neurotrophic factors to treat neurodegenerative disorders.

### SUMMARY

The present invention provides, in some embodiments, methods of treating mild to moderate Alzheimer's disease.
Embodiment 1. A method of treating mild to moderate Alzheimer's disease (AD), comprising administering to a patient in need thereof 2-90 mg per day of a compound of formula A19

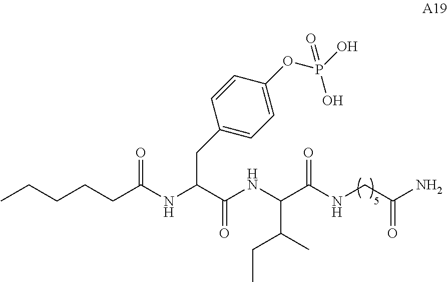

or a pharmaceutically acceptable salt thereof.
Embodiment 2. A method of treating mild AD, comprising administering to a patient in need thereof 2-90 mg per day of a compound of formula A19

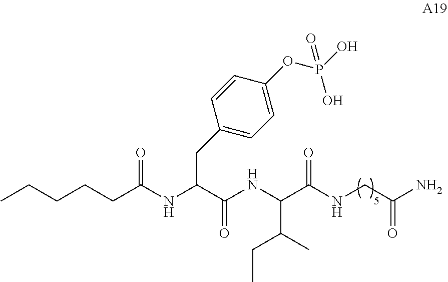

or a pharmaceutically acceptable salt thereof.
Embodiment 3. A method of treating moderate AD, comprising administering to a patient in need thereof 2-90 mg per day of a compound of formula A19

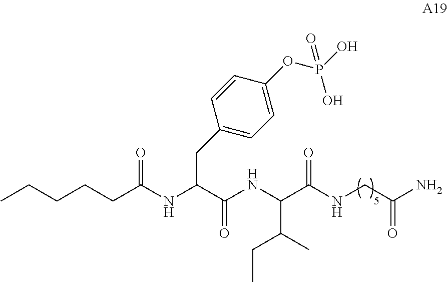

or a pharmaceutically acceptable salt thereof.
Embodiment 4. A method of slowing the decline in cognition or improving cognition in a patient, comprising administering to a patient in need thereof 2-90 mg per day of a compound of formula A19

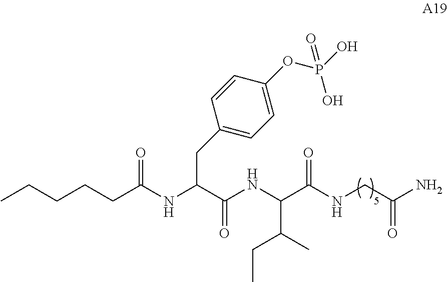

or a pharmaceutically acceptable salt thereof.
Embodiment 5. A method of slowing the decline in the ability to perform activities of daily living and verbal fluency or improving the ability to perform activities of daily living and verbal fluency in a patient diagnosed with mild to moderate AD, comprising administering to the patient 2-90 mg per day of a compound of formula A19

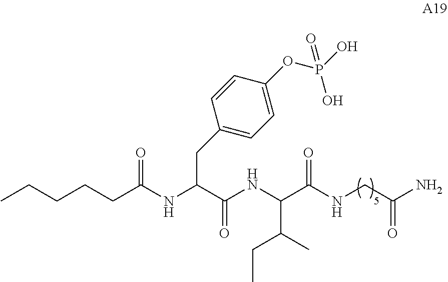

or a pharmaceutically acceptable salt thereof.
Embodiment 6. A method of slowing the decline in functional or cognitive capacity in a patient diagnosed with mild to moderate AD, comprising administering to the patient 2-90 mg per day of a compound of formula A19

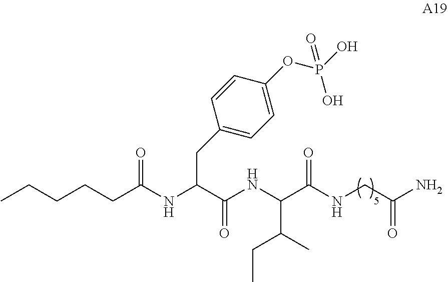

or a pharmaceutically acceptable salt thereof.
Embodiment 7. A method of slowing clinical decline in a patient diagnosed with mild to moderate AD, comprising administering to a patient diagnosed with mild to moderate AD 2-90 mg per day of a compound of formula A19

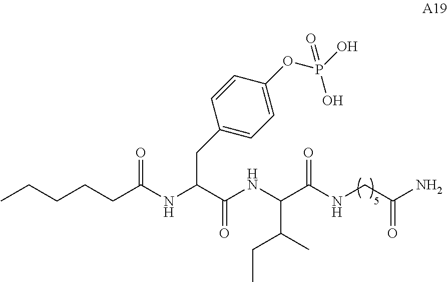

or a pharmaceutically acceptable salt thereof.
Embodiment 8. A method of improving executive memory function in a patient diagnosed with mild to moderate AD, comprising administering to a patient diagnosed with mild to moderate AD 2-90 mg per day of a compound of formula A19

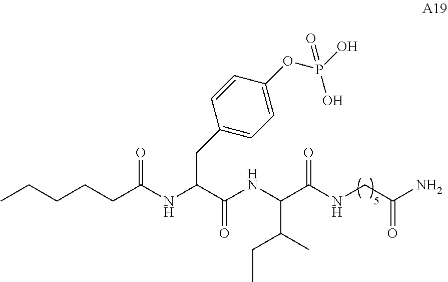

or a pharmaceutically acceptable salt thereof.
Embodiment 9. The method of any one of the preceding embodiments, wherein the patient has a Mini-Mental State Examination (MMSE) score of at least 14, between 14 and 24, between 15 and 24, between 16 and 24, between 17 and 24, between 18 and 24, between 19 and 24, between 20 and 24, between 21 and 24, between 22 and 24, between 23 and 24 prior to the start of treatment with the compound of formula A19.
Embodiment 10. The method of any one of the preceding embodiments, wherein the patient has been diagnosed with mild AD.
Embodiment 11. The method of any one of the preceding embodiments, wherein the patient has an MMSE score between 20 and 24 prior to the start of treatment with the compound of formula A19.
Embodiment 12. The method of any one of the preceding embodiments, wherein the patient has been diagnosed with moderate AD.
Embodiment 13. The method of any one of the preceding embodiments, wherein the patient has an MMSE score between 14 and 19 prior to the start of treatment with the compound of formula A19.
Embodiment 14. The method of any one of the preceding embodiments, wherein the patient has a Clinical Dementia Rating (CDR) Scale global score of 1 or 2 prior to the start of treatment with the compound of formula A19.
Embodiment 15. The method of any one of the preceding embodiments, wherein the patient is acetylcholinesterase inhibitor (AChEI) naïve or received an AChEI in the past, or received AChEI in the past and discontinued at least 4 weeks prior to administration of the compound.
Embodiment 16. The method of any one of the preceding embodiments, wherein the patient is between age 55 and 85.
Embodiment 17. The method of any one of the preceding embodiments, wherein the compound of formula A19 or the pharmaceutically acceptable salt thereof is administered by subcutaneous injection.
Embodiment 18. The method of any one of the preceding embodiments, comprising administering the compound of formula A19 or the pharmaceutically acceptable salt thereof at a dose of 2 mg, 10 mg, 20 mg, 30 mg, 40 mg, 50 mg, 60 mg, 70 mg, 80 mg, or 90 mg.
Embodiment 19. The method of any one of the preceding embodiments, comprising administering the compound of formula A19 or the pharmaceutically acceptable salt thereof at a dose of 40 mg or 70 mg.
Embodiment 20. The method of any one of the preceding embodiments, comprising administering the compound of formula A19 or the pharmaceutically acceptable salt thereof at a dose of 40 mg.
Embodiment 21. The method of any one of the preceding embodiments, comprising administering the compound of formula A19 or the pharmaceutically acceptable salt thereof at a dose of 70 mg.
Embodiment 22. The method of any one of the preceding embodiments, comprising administering the compound of formula A19 or the pharmaceutically acceptable salt thereof for 26 weeks or more.
Embodiment 23. The method of any one of the preceding embodiments, which slows the decline in functional or cognitive capacity in the patient.
Embodiment 24. The method of any one of the preceding embodiments, which slows the decline in cognition in the patient.
Embodiment 25. The method of any one of the preceding embodiments, which improves cognition in the patient.
Embodiment 26. The method of any one of the preceding embodiments, which slows the decline in the ability to perform activities of daily living and verbal fluency in the patient.
Embodiment 27. The method of any one of the preceding embodiments, which improves the ability to perform activities of daily living and verbal fluency in the patient.
Embodiment 28. The method of any one of embodiments 23-27, wherein the slowing of the decline or the improvement is determined after administering the treatment for at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks.
Embodiment 29. The method of any one of the preceding embodiments, wherein cognitive capacity is assessed by determining the patient's score before and after administration of the compound of formula A19 or the pharmaceutically acceptable salt thereof using an 11-item Alzheimer's Disease Assessment Scale—cognitive subscale (ADAS-Cog11).
Embodiment 30. The method of embodiment 23, wherein cognitive capacity is assessed prior to the start of treatment and at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.
Embodiment 31. The method of any one of the preceding embodiments, which reduces the rate of decline, stabilizes, or improves ADAS-Cog11.
Embodiment 32. The method of any one of the preceding embodiments, which reduces the rate of decline, stabilizes, or improves Alzheimer's Disease Cooperative Study-Clinical Global Impression of Change (ADCS-CGIC).
Embodiment 33. The method of embodiment 31 or embodiment 32, wherein reducing the rate of decline, stabilizing, or improving is assessed by determining the patient's score prior to the start of treatment and at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.
Embodiment 34. The method of any one of embodiments 29-33, wherein the onset of the effect on ADAS-Cog11 and/or ADCS-CGIC begins by 6 weeks, or by 8 weeks, or by 10 weeks, or by 12 weeks, or by 14 weeks, or by 16 weeks, or by 18 weeks, or by 20 weeks, or by 22 weeks, or by 24 weeks, or by 26 weeks after the start of treatment.
Embodiment 35. The method of any one of embodiments 29-34, wherein the effect on ADAS-Cog11 and/or ADCS-CGIC is maintained for at least 2 weeks or at least 4 weeks after the end of treatment.
Embodiment 36. The method of any one of the preceding embodiments, which reduces the rate of decline, stabilizes, or improves at least one, at least two, at least three, at least four, at least five, at least six, at least seven, at least eight, at least nine, or at least ten subdomains of a Neuropsychiatric inventory (NPI).
Embodiment 37. The method of any one of the preceding embodiments, which reduces the rate of decline, stabilizes, or improves a NPI score, Alzheimer's disease cooperative study-activities of daily living, 23-item version (ADCS-ADL23) score and/or Controlled Oral Word Association Test (COWAT) score.
Embodiment 38. The method of embodiment 37, wherein the reduction in the rate of decline, stabilization, or improvement occurs by at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.
Embodiment 39. The method of any one of embodiment 36-38, wherein reducing the rate of decline, stabilizing, or improving is assessed by determining the patient's score prior to the start of treatment and at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.
Embodiment 40. The method of any one of the preceding embodiments, which reduces the rate of decline, stabilizes, or improves a Resource utilization in dementia lite version (RUD-lite) scale, a EQ-5D-5L score, and/or Zarit burden interview (ZBI) score.
Embodiment 41. The method of embodiment 40, wherein the reduction in the rate of decline, stabilization, or improvement occurs by at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.
Embodiment 42. The method of embodiment 40 or embodiment 41, wherein reducing the rate of decline, stabilizing, or improving is assessed by determining the patient's score prior to the start of treatment and at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.
Embodiment 43. The method of any one of the preceding embodiments, which provides fast improvement or normalization of P300 values.
Embodiment 44. The method of any one of the preceding embodiments, which provides fast improvement or normalization of EPR P300 latency values.
Embodiment 45. The method of any one of the preceding embodiments, which provides fast improvement or normalization of ERP P300 latency values with some maintenance of effect at 4 weeks after discontinuation of treatment.
Embodiment 46. The method of any one of the preceding embodiments, which provides fast improvement or normalization of ERP P300 latency values, which is maintained at 4 weeks after discontinuation of treatment.
Embodiment 47. The method of any one of the preceding embodiments, which improves event-related potential (ERP) P300 latency.
Embodiment 48. The method of any one of embodiments 43-47, wherein the improvement or normalization occurs by at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.
Embodiment 49. The method of any one of the preceding embodiments, which improves quantitative EEG (qEEG).
Embodiment 50. The method of any one of the preceding embodiments, which improves qEEG at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.
Embodiment 51. The method of any one of the preceding embodiments, which improves serum NFL and/or neurogranin levels.
Embodiment 52. The method of any one of the preceding embodiments, which improves serum NFL and/or neurogranin levels, and at least one of phospho-tau, ABeta 1-42, and/or YLK41 levels.
Embodiment 53. The method of any one of the preceding embodiments, which has an acceptable safety and tolerability profile.
Embodiment 54. The method of any one of the preceding embodiments, which is generally safe and well tolerated.
Embodiment 55. The method of any one of the preceding embodiments, comprising administering a sodium salt of the compound of formula A19.
Embodiment 56. The method of any one of the preceding embodiments, comprising administering a monosodium salt of the compound of formula A19.
Embodiment 57. The method of any one of the preceding embodiments, comprising administering ATH-1017.

### BRIEF DESCRIPTION OF THE FIGURES

FIG. 1A shows P300 latency change from baseline in the study population, grouped by patients receiving placebo with and without AChEI therapy and patients receiving fosgonimeton (ATH-1017) with and without AChEI therapy.

FIG. 1B shows P300 latency change from baseline in a portion of the study population, grouped by patients receiving placebo with AChEI therapy and patients receiving fosgonimeton (ATH-1017) with AChEI therapy.

FIG. 1C shows P300 latency change from baseline in a portion of the study population, grouped by patients receiving placebo without AChEI therapy and patients receiving fosgonimeton (ATH-1017) without AChEI therapy.

FIG. 2A shows ADAS-Cog score change from baseline in the study population, grouped by patients receiving placebo with and without AChEI therapy, and patients receiving fosgonimeton (ATH-1017) with and without AChEI therapy.

FIG. 2B shows ADAS-Cog score change from baseline in a portion of the study population, grouped by patients receiving placebo with AChEI therapy, and patients receiving fosgonimeton (ATH-1017) with AChEI therapy.

FIG. 2C shows ADAS-Cog score change from baseline in a portion of the study population, grouped by patients receiving placebo without AChEI therapy, and patients receiving fosgonimeton (ATH-1017) without AChEI therapy.

### DETAILED DESCRIPTION

ATH-1017 is an experimental Alzheimer's disease (AD) treatment, formulated as a sterile solution for subcutaneous (SC) injection. ATH-1017 is a prodrug, which is rapidly converted to the active drug ATH-1001 (Dihexa; see US2014/0094413) in the plasma after SC injection. ATH-1017 was developed as a water-soluble prodrug of ATH-1001 to allow SC dosing in aqueous vehicles. The active drug ATH-1001 acts as an agonist of the hepatic growth factor (HGF) receptor and its tyrosine kinase, MET. Central nervous system (CNS) MET expression is crucial in maintaining the healthy adult brain (Hawrylycz, 2015), and is reduced in AD, particularly in the hippocampus (Hamasaki, 2014). The HGF/MET system presents a therapeutic target to treat neurodegeneration and restore cognitive function in AD.

### Definitions and General Parameters

As used in the present specification, the following terms and phrases are generally intended to have the meanings as set forth below, except to the extent that the context in which they are used indicates otherwise.

Abbreviations that may be used in this description:

| Abbreviation | Definition |
|---|---|
| AChEI | Acetylcholinesterase inhibitor |
| AD | Alzheimer's disease |
| AE | Adverse event |
| ADAS-Cog₁₁ | Alzheimer's disease assessment scale-cognitive subscale |
| ADCS-ADL23 | Alzheimer's disease cooperative study-activities of |
| | daily living, 23-item version |
| ADCS-CGIC | Alzheimer's disease cooperative study-clinical global |
| | impression of change |
| AKT | Protein kinase B |
| ALP | Alkaline phosphatase |
| ALT | Alanine aminotransferase |
| ApoE | Apolipoprotein E |
| aPTT | Activated partial thromboplastin time |
| AST | Aspartate aminotransferase |
| CBC | Complete blood count |
| CBD | Cannabidiol |
| CDR | Clinical dementia rating scale |
| CPK | Creatine phosphokinase |
| CRO | Contract research organization |
| CYP3A4 | Cytochrome P450 3A4 |
| Cₘₐₓ | Maximum concentration |
| CNS | Central nervous system |
| COWAT | Controlled oral word association test |
| C-SSRS | Columbia-suicide severity rating scale |
| CT | Computerized tomography |
| DSMB | Data safety monitoring board |
| ECG | Electrocardiogram |
| eCRF | Electronic case report form |
| EDC | Electronic data capture |
| EEG | Electroencephalogram |
| EQ-5D-5L | EuroQol group 5-dimension 5 level questionnaire |
| ERP | Event-related potentials |
| ET | Early termination |
| FSH | Follicle-stimulating hormone |
| fT3 | free tri-iodothyronine |
| fT4 | free thyroxine |
| FWER | Family-wise error rate |
| GCP | Good clinical practice |
| GDS | Geriatric depression scale |
| GGT | Gamma-glutamyl transferase |
| GLP | Good laboratory practice |
| GST | Global statistical test |
| HBsAg | Hepatitis B surface antigen |
| HCV | Hepatitis C virus |
| HGF | Hepatic growth factor |
| HIV | Human immunodeficiency virus |
| HR | Heart rate |
| ICF | Informed consent form |
| ICH | International council for harmonisation |
| IEC | Independent ethics committee |
| INR | International normalized ratio |
| IRB | Institutional review board |
| IRT | Interactive response technology |
| LAR | Legally authorized representative |
| LTP | Long-term potentiation |
| MAPK | Mitogen-activated protein kinase |
| MCT | Medium-chain triglyceride |
| MET | MET receptor tyrosine kinase |
| mITT | Modified intent-to-treat |
| MMRM | Mixed model for repeated measures |
| MMSE | Mini-mental state examination |
| MRI | Magnetic resonance imaging |
| NMDA | N-methyl D-aspartate |
| NPI | Neuropsychiatric inventory |
| OD | Once-daily |
| P | Phosphorylated |
| PD | Pharmacodynamic(s) |
| PI3K | Phosphoinositide 3-kinase |
| PK | Pharmacokinetic(s) |
| PKC | Protein kinase C |
| PLCγ | Phospholipase C-gamma |
| PM | Plasma membrane |
| PRN | As needed |
| PSP | Post-synaptic potential |
| PT | Prothrombin time |
| qEEG | Quantitative electroencephalogram |
| QTcF | Corrected QT interval using Fridericia's formula |
| RAC1 | Ras-related C3 botulinum toxin substrate 1 |
| RAF | Rapidly accelerated fibrosarcoma (protein) |
| RAS | Rat sarcoma (protein) |
| RBC | Red blood cells |
| RUD-Lite ® | Resource utilization in dementia lite version |
| SAE | Serious adverse event |
| SAP | Statistical analysis plan |
| SBP | Systolic blood pressure |
| SC | Subcutaneous |
| SOP | Standard operating procedure |
| STAT3 | Signal transducer and activator of transcription 3 |
| THC | Tetrahydrocannabinol |
| TSH | thyroid-stimulating hormone |
| ULN | Upper limit of normal |
| US(A) | United States (of America) |
| VAS | Visual analog scale |
| WBC | White blood cells |
| ZBI | Zarit burden interview |

Reference to “about” a value or parameter herein includes (and describes) embodiments that are directed to that value or parameter per se. In certain embodiments, the term “about” includes the indicated amount ±10%. In other embodiments, the term “about” includes the indicated amount ±5%. In certain other embodiments, the term “about” includes the indicated amount ±1%. Also, to the term “about X” includes description of “X”. Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

The disclosures illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure claimed.

In some embodiments, the compounds of the present disclosure can be in the form of a “prodrug.” The term “prodrug” is defined in the pharmaceutical field as a biologically inactive derivative of a drug that upon administration to the human body is converted to the biologically active parent drug according to some chemical or enzymatic pathway. Examples of prodrugs include esterified carboxylic acids.

The compounds of the present disclosure can be in the form of a pharmaceutically acceptable salt. The term “pharmaceutically acceptable salts” refers to salts prepared from pharmaceutically acceptable non-toxic bases or acids, including inorganic bases or acids and organic bases or acids. In case the compounds of the present disclosure contain one or more acidic or basic groups, the disclosure also comprises their corresponding pharmaceutically or toxicologically acceptable salts, in particular their pharmaceutically utilizable salts. Thus, the compounds of the present disclosure which contain acidic groups can be present on these groups and can be used according to the disclosure, for example, as alkali metal salts, alkaline earth metal salts or ammonium salts. More precise examples of such salts include sodium salts, potassium salts, calcium salts, magnesium salts or salts with ammonia or organic amines such as, for example, ethylamine, ethanolamine, triethanolamine, amino acids, or other bases known to persons skilled in the art. The compounds of the present disclosure which contain one or more basic groups, i.e., groups which can be protonated, can be present and can be used according to the disclosure in the form of their addition salts with inorganic or organic acids.

The present disclosure provides pharmaceutical compositions comprising a compound of the present disclosure, or a pharmaceutically acceptable salt or solvate thereof as active ingredient together with a pharmaceutically acceptable carrier.

“Pharmaceutical composition” means one or more active ingredients, and one or more inert ingredients that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present disclosure can encompass any composition made by admixing at least one compound of the present disclosure and a pharmaceutically acceptable carrier.

As used herein, “pharmaceutically acceptable carrier” includes excipients or agents such as solvents, diluents, dispersion media, coatings, antibacterial and antifungal agents, isotonic and absorption delaying agents and the like that are not deleterious to the disclosed compound or use thereof. The use of such carriers and agents to prepare compositions of pharmaceutically active substances is well known in the art (see, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.).

The terms “therapeutically effective amount” and “effective amount” are used interchangeably and refer to an amount of a compound that is sufficient to effect treatment as defined herein, when administered to a patient (e.g., a human) in need of such treatment in one or more doses. The therapeutically effective amount will vary depending upon the patient, the disease being treated, the weight and/or age of the patient, the severity of the disease, or the manner of administration as determined by a qualified prescriber or care giver.

The term “treatment” or “treating” means administering a compound or pharmaceutically acceptable salt thereof for the purpose of: (i) delaying the onset of a disease, that is, causing the clinical symptoms of the disease not to develop or delaying the development thereof; (ii) inhibiting the disease, that is, arresting the development of clinical symptoms; and/or (iii) relieving the disease, that is, causing the regression of clinical symptoms or the severity thereof.

### ATH-1017 and Related Compounds

ATH-1017 is a pharmaceutically acceptable salt of the compound having the formula of A19:

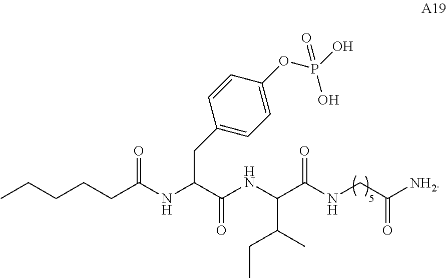

As used herein, and in the absence of a specific reference to a particular pharmaceutically acceptable salt and/or solvate of ATH-1017, any dosages, whether expressed in e.g. milligrams or as a % by weight, should be taken as referring to the amount of ATH-1017, i.e. the amount of:

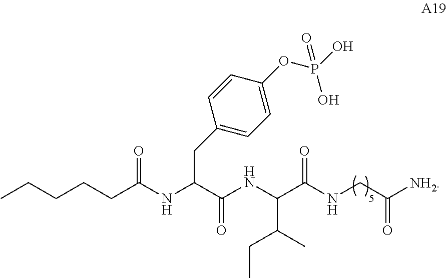

For example, therefore, a reference to “40 mg ATH-1017 or a pharmaceutically acceptable salt and/or solvate thereof” means an amount of ATH-1017 or a pharmaceutically acceptable salt and/or solvate thereof which provides the same amount of ATH-1017 as 40 mg of A19 free acid.

Nonlimiting exemplary pharmaceutically acceptable salts of A19 include:

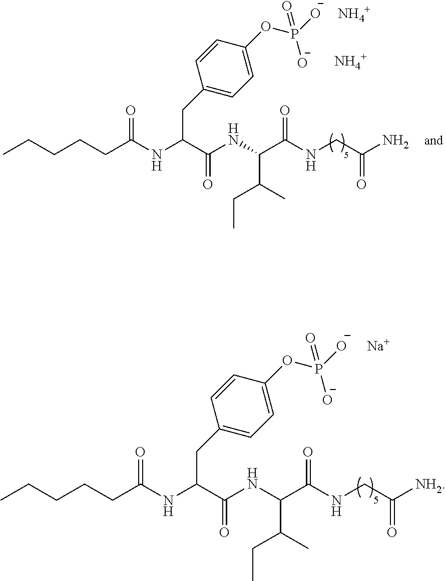

Unless otherwise indicated, ATH-1017 refers to the monosodium salt of A19, shown below:

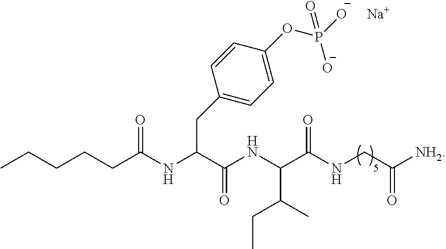

The compound of A19, and pharmaceutically acceptable salts thereof, including ATH-1017, may be synthesized and characterized using methods known to those of skill in the art, such as those described in PCT Publication No. WO 2017/210489 A1.

In some embodiments, ATH-1017 is formulated for subcutaneous administration. In some such embodiments, ATH-1017 is provided in a pre-filled syringe containing 1 mL of 40 mg/mL ATH-1017 or 70 mg/mL ATH-1017. In some embodiments, the ATH-1017 is in a solution comprising 10 mM sodium phosphate.

### Methods of Treating Alzheimer's Disease

Provided herein are methods of treating mild to moderate Alzheimer's Disease (AD), comprising administering to a patient a therapeutically effective amount of ATH-1017.

In some embodiments, a patient with mild to moderate AD is defined as a patient with a Mini-Mental State Examination (MMSE) score of 14 to 24. In some embodiments, a patient with moderate AD is defined as a patient with a MMSE score of 14 to 19. In some embodiments, a patient with mild AD is defined as a patient with a MMSE score of 20 to 24.

In some embodiments, a method of treating mild to moderate AD is provided, comprising administering to a patient in need thereof 2-90 mg per day of a compound of formula A19 or a pharmaceutically acceptable salt thereof, such as a sodium salt of the compound of formula A19, such as a monosodium salt of the compound of formula A19, such as ATH-1017.

In some embodiments, a method of treating mild AD is provided, comprising administering to a patient in need thereof 2-90 mg per day of a compound of formula A19 or a pharmaceutically acceptable salt thereof, such as a sodium salt of the compound of formula A19, such as a monosodium salt of the compound of formula A19, such as ATH-1017.

In some embodiments, a method of treating moderate AD is provided, comprising administering to a patient in need thereof 2-90 mg per day of a compound of formula A19 or a pharmaceutically acceptable salt thereof, such as a sodium salt of the compound of formula A19, such as a monosodium salt of the compound of formula A19, such as ATH-1017.

In some embodiments, a method of slowing the decline in cognition or improving cognition in a patient is provided, comprising administering to a patient in need thereof 2-90 mg per day of a compound of formula A19 or a pharmaceutically acceptable salt thereof, such as a sodium salt of the compound of formula A19, such as a monosodium salt of the compound of formula A19, such as ATH-1017.

In some embodiments, a method of slowing the decline in the ability to perform activities of daily living and verbal fluency or improving the ability to perform activities of daily living and verbal fluency in a patient diagnosed with mild to moderate AD is provided, comprising administering to a patient in need thereof 2-90 mg per day of a compound of formula A19 or a pharmaceutically acceptable salt thereof, such as a sodium salt of the compound of formula A19, such as a monosodium salt of the compound of formula A19, such as ATH-1017.

In some embodiments, a method of slowing the decline in functional or cognitive capacity in a patient diagnosed with mild to moderate AD is provided, comprising administering to a patient in need thereof 2-90 mg per day of a compound of formula A19 or a pharmaceutically acceptable salt thereof, such as a sodium salt of the compound of formula A19, such as a monosodium salt of the compound of formula A19, such as ATH-1017.

In some embodiments, a method of slowing clinical decline in a patient diagnosed with mild to moderate AD is provided, comprising administering to a patient in need thereof 2-90 mg per day of a compound of formula A19 or a pharmaceutically acceptable salt thereof, such as a sodium salt of the compound of formula A19, such as a monosodium salt of the compound of formula A19, such as ATH-1017.

In some embodiments, a method of improving executive memory function in a patient diagnosed with mild to moderate AD is provided, comprising administering to a patient in need thereof 2-90 mg per day of a compound of formula A19 or a pharmaceutically acceptable salt thereof, such as a sodium salt of the compound of formula A19, such as a monosodium salt of the compound of formula A19, such as ATH-1017.

In some embodiments, the patient has a Mini-Mental State Examination (MMSE) score of at least 14, between 14 and 24, between 15 and 24, between 16 and 24, between 17 and 24, between 18 and 24, between 19 and 24, between 20 and 24, between 21 and 24, between 22 and 24, between 23 and 24 prior to the start of treatment with the compound of formula A19 or a pharmaceutically acceptable salt thereof, such as a sodium salt of the compound of formula A19, such as a monosodium salt of the compound of formula A19, such as ATH-1017. In some embodiments, the patient has been diagnosed with mild AD. In some embodiments, the patient has an MMSE score between 20 and 24 prior to the start of treatment with the compound of formula A19. In some embodiments, the patient has been diagnosed with moderate AD. In some embodiments, the patient has an MMSE score between 14 and 19 prior to the start of treatment with the compound of formula A19 or a pharmaceutically acceptable salt thereof, such as a sodium salt of the compound of formula A19, such as a monosodium salt of the compound of formula A19, such as ATH-1017.

In some embodiments, the patient has a Clinical Dementia Rating (CDR) Scale global score of 1 or 2 prior to the start of treatment with the compound of formula A19 or a pharmaceutically acceptable salt thereof, such as a sodium salt of the compound of formula A19, such as a monosodium salt of the compound of formula A19, such as ATH-1017. In some embodiments, the patient is between age 55 and 85.

In some embodiments, the patient is acetylcholinesterase inhibitor (AChEI) naïve. In some embodiments, the patient received an AChEI in the past. In some embodiments, the patient discontinued AChEI therapy at least 4 weeks prior to administration of the compound.

In some embodiments, the compound of formula A19 or the pharmaceutically acceptable salt thereof, such as a sodium salt of the compound of formula A19, such as a monosodium salt of the compound of formula A19, such as ATH-1017, is administered by subcutaneous injection.

In some embodiments, the compound of formula A19 or the pharmaceutically acceptable salt thereof is administered at a dose of 2 mg, 10 mg, 20 mg, 30 mg, 40 mg, 50 mg, 60 mg, 70 mg, 80 mg, or 90 mg. In some embodiments, the compound of formula A19 or the pharmaceutically acceptable salt thereof is administered at a dose of 40 mg or 70 mg. In some embodiments, the compound of formula A19 or the pharmaceutically acceptable salt thereof, such as a sodium salt of the compound of formula A19, such as a monosodium salt of the compound of formula A19, such as ATH-1017, is administered at a dose of 40 mg. In some embodiments, the compound of formula A19 or the pharmaceutically acceptable salt thereof, such as a sodium salt of the compound of formula A19, such as a monosodium salt of the compound of formula A19, such as ATH-1017, is administered at a dose of 70 mg.

In some embodiments, the compound of formula A19 or the pharmaceutically acceptable salt thereof, such as a sodium salt of the compound of formula A19, such as a monosodium salt of the compound of formula A19, such as ATH-1017, is administered for 26 weeks or more.

In some embodiments, the methods of treatment described herein slow the decline in or improve functional or cognitive capacity in the patient. In some embodiments, the methods of treatment slow the decline in or improve cognition in the patient. In some embodiments, the methods of treatment slow the decline in or improve the ability to perform activities of daily living and verbal fluency in the patient.

In some embodiments, the methods of treatment described herein improve cognition in the patient. In some embodiments, the methods of treatment improve the ability to perform activities of daily living and verbal fluency in the patient.

In some embodiments, the slowing of the decline or the improvement is determined after administering the treatment for at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks.

Various methods are useful in assessing the cognitive capacity of the patient.

In some embodiments, cognitive capacity is assessed by determining the patient's score before and after administration of the compound of formula A19 or the pharmaceutically acceptable salt thereof using an 11-item Alzheimer's Disease Assessment Scale—cognitive subscale (ADAS-Cog11). In some embodiments, cognitive capacity is assessed prior to the start of treatment and at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment. In some embodiments, the method of treatment reduces the rate of decline, stabilizes, or improves in ADAS-Cog11 or improve ADAS-Cog11.

In some embodiments, the method of treatment reduces the rate of decline, stabilizes, or improves Alzheimer's Disease Cooperative Study-Clinical Global Impression of Change (ADCS-CGIC).

In some embodiments, the method of treatment reduces the rate of decline, stabilizes, or improves cognition as measured by the Global Statistical Test (GST), which combines the ADAS-Cog11 and ADCS-CGIC.

In some embodiments, reducing the rate of decline, stabilizing, or improving is assessed by determining the patient's score prior to the start of treatment and at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.

In some embodiments, the onset of the effect on ADAS-Cog11 and/or ADCS-CGIC begins by 6 weeks, or by 8 weeks, or by 10 weeks, or by 12 weeks, or by 14 weeks, or by 16 weeks, or by 18 weeks, or by 20 weeks, or by 22 weeks, or by 24 weeks, or by 26 weeks after the start of treatment.

In some embodiments, the effect on ADAS-Cog11 and/or ADCS-CGIC is maintained for at least 2 weeks or at least 4 weeks after the end of treatment.

In some embodiments, the method of treatment reduces the rate of decline, stabilizes, or improves at least one, at least two, at least three, at least four, at least five, at least six, at least seven, at least eight, at least nine, or at least ten subdomains of Neuropsychiatric inventory (NPI) score. In some embodiments, the method of treatment reduces the rate of decline, stabilizes, or improves a NPI score, Alzheimer's disease cooperative study-activities of daily living, 23-item version (ADCS-ADL23) score and/or Controlled Oral Word Association Test (COWAT) score. In some embodiments, the method of treatment reduces the rate of decline, stabilizes, or improves a in NPI score, ADCS-ADL23 score and/or COWAT score at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.

In some embodiments, the NPI score at week 26 after the start of treatment is lower than an NPI score before treatment started or a difference is statistically significant between an NPI score at week 26 after the start of treatment and an NPI score before treatment started.

In some embodiments, the ADCS-ADL23 score improves by at least 2 points or at least one point.

In some embodiments, the COWAT score at week 26 after the start of treatment is higher than a COWAT score before treatment started or a difference is statistically significant between a COWAT score at week 26 after the start of treatment and a COWAT score before treatment started.

In some embodiments, the method of treatment reduces the rate of decline, stabilizes, or improves a in Resource utilization in dementia lite version (RUD-lite) scale, a EQ-5D-5L score, and/or Zarit burden interview (ZBI) score. In some embodiments, the method of treatment reduces the rate of decline, stabilizes, or improves RUD-lite scale, EQ-5D-5L score, and/or ZBI score at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.

In some embodiments, the RUD-lite scale, an EQ-5D-5L score, and/or Zarit burden interview score improve by a statistically significant amount at week 26 after the start of treatment.

In some embodiments, the methods of treatment provide fast improvement or normalization of P300 values or ERP P300 latency values. In some embodiments, the methods of treatment provide fast improvement or normalization of P300 values or ERP P300 latency values with some maintenance of effect at 4 weeks after discontinuation of treatment.

In some embodiments, the methods of treatment improve event-related potential (ERP) P300 latency. In some embodiments, the methods of treatment improve ERP P300 latency at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.

In some embodiments, ERP P300 latency improves by at least 40 milliseconds or at least 30 milliseconds or at least 20 milliseconds.

In some embodiments, the methods of treatment improve quantitative EEG (qEEG). In some embodiments, the methods of treatment improve qEEG at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.

In some embodiments, the methods of treatment improve serum NFL and/or neurogranin levels. In some embodiments, the methods of treatment improve NFL and/or neurogranin levels, and at least one of phospho-tau, ABeta 1-42, and/or YLK41 levels.

In some embodiments, the methods of treatment have an acceptable safety and tolerability profile. In some embodiments, the methods of treatment are generally safe and well tolerated.

### P300 Latency and Gamma Power

Event-related potential (ERP) P300 latency is a functional measure of working memory processing speed and executive function that highly correlates with cognition. In some embodiments, administration of ATH-1017 in AD subjects significantly improves P300 latency. In some embodiments, after a single dose of ATH-1017, the AD subject has improved P300 latency. In some embodiments, by the end of an 8-day treatment cycle, average ERP P300 latency across the AD treatment group improves by 73 milliseconds compared to placebo group. In some embodiments, this improvement is a statistically significant change compared to placebo group. These results suggest that ATH-1017 has the potential to substantially improve synaptic connectivity and as a consequence, brain function in AD subjects.

Gamma power is typically associated with learning, memory, and cognitive function. In some embodiments, administration of ATH-1017 increases high frequency gamma power activity in AD subjects.

### Pharmaceutical Compositions

In some embodiments, the method includes administering ATH-1017 by subcutaneous injection.

Pharmaceutical compositions for the drugs provided herein may be in a form suitable for the administration routes. The formulations can conveniently be presented in unit dosage form and may be prepared by any of the methods well known in the art of pharmacy. Techniques and formulations generally are found in Remington's Pharmaceutical Sciences (Mack Publishing Co., Easton, Pa.).

The pharmaceutical compositions of the disclosure may be in the form of a sterile injectable preparation, such as, for example, a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent. Among the acceptable vehicles and solvents that may be employed are water, Ringer's solution and isotonic sodium chloride solution. In addition, sterile fixed oils may conventionally be employed as a solvent or suspending medium. For this purpose, any bland fixed oil may be employed including synthetic mono- or diglycerides. In addition, fatty acids such as, for example, oleic acid may likewise be used in the preparation of injectables.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

The formulations can be presented in unit-dose or multi-dose containers, for example sealed ampoules and vials, and may be stored in a freeze-dried (lyophilized) condition requiring only the addition of the sterile liquid carrier, for example water for injection, immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

The amount of active ingredient that may be combined with the carrier material to produce a single dosage form will vary depending upon the host treated and the particular mode of administration, such as subcutaneous injection. The pharmaceutical composition can be prepared to provide easily measurable amounts for administration. For example, in some embodiments, ATH-1017 is formulated for subcutaneous administration, provided in a pre-filled syringe containing 1 mL of 40 mg/mL ATH-1017 or 70 mg/mL ATH-1017. In some embodiments, the ATH-1017 is in a solution comprising 10 mM sodium phosphate.

### EXAMPLES

The following examples are included to demonstrate specific embodiments of the disclosure. It should be appreciated by those of skill in the art that the techniques disclosed in the examples which follow represent techniques to function well in the practice of the disclosure, and thus can be considered to constitute specific modes for its practice. However, those of skill in the art should, in light of the present disclosure, appreciate that these examples are exemplary and not exhaustive. Many changes can be made in the specific embodiments which are disclosed and still obtain a like or similar result without departing from the spirit and scope of the disclosure.

### Example 1: Clinical Study Evaluating ATH-1017 in Subjects with Mild to Moderate Alzheimer's Disease

### A. Overview of Study Design

In human clinical studies of ATH-1017, single SC administration of 2, 6, 20, 40, 60, and 90 mg in healthy young subjects, and multiple administration of 20, 40, 60, and 80 mg (SC, OD, over 9 consecutive days) in healthy elderly subjects, and 40 mg (SC, OD, over 9 consecutive days) in AD subjects were safe and well tolerated. Injection site reactions included pain, pruritus, and/or erythema, were mild in nature, and resolved without specific therapy. A potential risk for hepatotoxicity identified in nonclinical studies has not been observed in human studies but are closely monitored in this study. To date, no CNS-specific adverse events have been observed in humans.

This is a Phase 2/3 multicenter, randomized, double-blind, placebo-controlled, parallel-group, dose-ranging study comparing ATH-1017 40 mg/day and ATH-1017 70 mg/day with placebo in subjects with a diagnosis of mild to moderate Alzheimer's disease (AD), diagnosed on a ‘probable’ level according to McKhann 2011. The study is conducted at a total of approximately 55 centers in the US (plus Australia, optionally). Subjects and their caregivers are required to sign an informed consent form (ICF) and are evaluated against the inclusion/exclusion criteria during a screening period. Those who meet all inclusion/exclusion criteria are randomized in a ratio of 1:1:1 to 3 parallel arms, either to active treatment (ATH-1017 40 mg/day or ATH-1017 70 mg/day) or placebo. During randomization, subjects are stratified by screening Mini-Mental State Examination (MMSE) severity: mild (MMSE: 20-24) versus moderate (MMSE: 14-19). All eligible subjects are tested for apolipoprotein E (ApoE) genotype.

Study drugs are administered by subcutaneous (SC) injection once-daily (OD) preferably during daytime. The first SC injection of study drug are performed at site under supervision. The subject should withhold study drug administration on the day of subsequent clinic visits; study drug administration is done on site under supervision of site staff at these visits. Each subject is required to have a primary caregiver willing to accept responsibility for supervising or, if required, administering study drug, and assessing the condition of the subject throughout the study in accordance with all protocol requirements.

The study consists of up to 28 days of screening (Day −28 through Day −1) followed by 26 weeks of double-blind treatment and a 4-week safety follow-up. During the double-blind treatment period, clinic visits take place on Day 1 and thereafter at Weeks 2, 6, 12, 16, 20, and 26, with a safety follow-up visit scheduled 4 weeks after completion of the double-blind period at Week 30. On Day 1, after completion of the first dose, subjects remain on-site 2 hours for post-treatment clinical observation. As marked circadian fluctuations of cognitive performance have been observed in AD (Hilt, 2015), ADAS-Cog11 and COWAT assessments shall occur at clinic visits in the morning at approximately the same time they were performed during the initial Baseline assessment. Similarly, ADCS-CGIC assessments are organized adjacent to the individual ADASCog11 and COWAT assessment times. Subjects may live at home, in a senior residential setting, or an institutional setting without the need for continuous nursing care, and should not be likely to experience a change in living conditions (e.g., institutionalization, moving to a different city, etc.), or change in primary caregiver, during participation in the trial period. The end of the study is defined as the date of the safety follow-up visit, Visit 9/Week 30. Subjects who terminate prior to Visit 8 are to complete same assessments as Visit 8/early termination (ET).

Blood draws take place at scheduled clinic visits (Day 1, Week 12 and Week 26) for analysis of plasma concentrations of ATH-1017 and ATH-1001.

An independent Data Safety Monitoring Board conducts periodic review and assessments of unblinded safety data (AEs, labs, ECG, etc.) throughout the study to ensure the safety of study subjects.

An optional 26-week open-label extension study is offered at participating sites for subjects who complete the 26-week randomized study.

### B. Subject Population

The study randomizes up to approximately 300 in a 1:1:1 ratio to ATH-1017 40 mg, ATH-1017 70 mg, and placebo groups subjects in order to include a total of approximately 240 evaluable subjects in the analysis of the primary endpoint.

### Inclusion Criteria

Subjects must meet all of the following inclusion criteria to participate in the study.
1) Age 55 to 85 years, inclusive at the time of signing the informed consent.
2) Mild-to-moderate AD dementia subjects.
   a) MMSE score 14 to 24 inclusive at Screening
   b) Clinical Dementia Rating (CDR) Scale global score of 1 or 2 at Screening
3) Clinical diagnosis of dementia, due probably to AD, by Revised National Institute on Aging-Alzheimer's Association criteria (McKhann 2011):
   a) Magnetic resonance imaging (MM) or computerized tomography (CT) scan (for subjects with non-MRI-safe cardiac pacemaker, or other relevant medical reason, with Medical Monitor approval) performed within 12 months before Screening, with findings consistent with the diagnosis of dementia due to AD without any other significant comorbid central nervous system pathologies. If such scan is unavailable or older than 12 months, it should be repeated to ascertain the diagnosis before randomization.
   b) Documented clinical decline within 12 months before Screening and onset of symptoms at least 12 months before Screening (preferably subject medical records; caregiver reports with examples are acceptable).
4) Formal education of 8 or more years; exceptions may be made for subjects with less than 8 years of education at the discretion of the investigator.
5) Body mass index (BMI) of ≥18 and ≤35 kg/m2 at Screening; subjects with BMI outside the allowed BMI range but ≥16 and ≤37 kg/m2 may enroll only with prior agreement of the Sponsor.
6) Male subjects and their partners must agree to use a double-barrier method of contraception during the study, including the follow-up period, unless the partner is not of childbearing potential. Only female subjects of non-childbearing potential (i.e., permanently sterilized, postmenopausal) are eligible for participation.
7) Reliable and capable support person/caregiver, who is willing to accept responsibility for supervising the treatment or, if required, administering study drug and assessing the condition of the subject throughout the study in accordance with all protocol requirements. The support person/caregiver must see the subject at least once-daily for dose administration and/or observation and have approximately 4 to 6 hours daytime contact with the subject for at least 4 days/week.
8) Treatment-free or receiving stable acetylcholinesterase inhibitor (AChEI) treatment, defined as:
   a) Treatment-naïve, OR
   b) Concomitant therapy with AChEI is allowed as long as the dose has been stable for 3 months prior to Screening (cf. EC #23b) and no changes are planned during the study, OR
   c) Subjects who received an AChEI in the past and discontinued 4 weeks prior to Screening.
9) Subject capable of giving signed informed consent, which includes compliance with the requirements and restrictions listed in the ICF and in this protocol. If the subject is incapable of giving informed consent in the judgment of the investigator then consent may be provided by a legally acceptable representative.
10) Written informed consent from a) the subject or legally acceptable representative and b) caregiver/support person has been obtained prior to any study-related procedures, including prior to initiating procedures to evaluate eligibility for the study.
11) Written documentation has been obtained in accordance with the relevant country and local privacy requirements, where applicable (e.g., Written Authorization for Use and Release of Health and Research Study Information).
12) Subjects and caregivers/support persons are able, as assessed by the investigator, and willing to follow study instructions and likely to complete all required study visits.
13) Subjects must be in generally good health as assessed by the investigator from medical history and physical/neurological examination, vital signs, ECG, and standard laboratory tests.

### Exclusion Criteria

Subjects who meet any of the following criteria are excluded from the study:
1) History of significant neurologic disease, other than AD, that may affect cognition, or concurrent with the onset of dementia.
2) Subject has atypical variant presentation of AD, if known from medical history, particularly non-amnestic AD.
3) History of brain MRI scan indicative of any other significant abnormality, including but not limited to multiple (>10) microhemorrhages, severe white matter hyperintensities, history or evidence of a single prior hemorrhage >1 cm3, multiple (>3) lacunar infarcts or evidence of a single prior infarct >1 cm3, evidence of a cerebral contusion, encephalomalacia, aneurysms, vascular malformations, subdural hematoma, or space-occupying lesions (e.g., brain tumors). If a known meningioma has been stable for >1 year and the subject has no history of any type of convulsions, this can be allowable after consultation with the Medical Monitor. Note: a new MM scan is required if the scan was performed >12 months prior to Screening; a repeat MRI scan is required if there have been intervening changes to the subject's clinical presentation in the past 12 months. CT scan is acceptable for subjects fitted with non-MRI-safe cardiac pacemaker or other relevant medical reason, with Medical Monitor approval.
4) Diagnosis with current symptoms of severe major depressive disorder even without psychotic features. Any subject with formalized delusions or hallucinations are excluded.
5) GDS score (15-item scale)>7 at Screening. In discussion with the Medical Monitor, subjects with a GDS score between 8 and 10 inclusive can be considered for study participation if the increased score is driven by specific domains related to the pandemic and its restrictions, rather than by major depression.
6) Significant suicide risk as defined by suicidal ideation based on the C-SSRS within the last 12 months, at Screening and on Day 1 (i.e., a ‘yes’ response to Question 4 or 5, or any specific behaviors).
7) History within 2 years of Screening, or current diagnosis of psychosis (American Psychiatric Association 2000) or moderate substance abuse disorder (according to the Diagnostic and Statistical Manual of Mental Disorders, Fifth Edition).
8) Untreated conditions, including vitamin B12 or folate deficiency, hypothyroidism, diabetes mellitus, hypo- or hypertension, if clinically relevant in the judgment of the investigator. If treated, must be stably treated and symptom-free for at least 6 months before Screening.
9) Abnormal serum electrolytes (potassium, sodium, magnesium) of clinical significance. If treated, must be stably treated for at least 30 days before Screening.
10) Active, acute, or chronic infectious disease of any type.
11) Myocardial infarction or unstable angina within the last 6 months or history of more than one myocardial infarction within 5 years before Screening.
12) Clinically significant (in the judgment of the investigator) cardiac arrhythmia (including atrial fibrillation), cardiomyopathy, or cardiac conduction defect (note: pacemaker is acceptable).
13) Subject has either hypertension (supine diastolic blood pressure >95 mmHg), or symptomatic hypotension in the judgment of the investigator.
14) Clinically significant ECG abnormality at Screening, including but not limited to a confirmed corrected QT interval using Fridericia's formula (QTcF) value ≥450 msec for males and ≥470 msec for females. For QTcF readings that are borderline, or difficult to interpret due to e.g., presence of a Branch Bundle Block, or in those where the T and U waves are superimposed or connected, a manual, local reading using the same ECG device should be considered to determine eligibility, in discussion with the Medical Monitor. In subjects with a QRS value 120 msec, those with a QTcF value <500 msec may be eligible following discussion with the Medical Monitor.
15) History of or positive results of serology screening for hepatitis B (hepatitis B surface antigen), hepatitis C (anti-hepatitis C virus antibodies) or human immunodeficiency virus (antibodies type 1 and 2).
16) Renal insufficiency (serum creatinine >2.0 mg/dL).
17) Hepatic impairment with alanine aminotransferase or aspartate aminotransferase >2 times the upper limit of normal, or Child-Pugh class B and C.
18) Malignant tumor within 3 years before Screening, except for the following conditions that are stable in the judgement of the Investigator
   a) Adequately treated squamous and basal cell carcinoma, or squamous and basal cell carcinoma in situ;
   b) Prostate carcinoma in situ.
19) Clinically significant (in the judgment of the investigator) unintentional weight loss within 12 months of Screening.
20) The consumption of grapefruit or grapefruit-containing products is prohibited beginning 7 days prior to the first dose of study medication (Day 1) and during the study.
21) Food supplements and nutraceuticals with potential effects on cognition, such as Axona and medium-chain triglyceride, are prohibited beginning 7 days prior to the first dose of study medication (Day 1) and for the duration of the study.
22) Tetrahydrocannabinol (THC) is prohibited beginning 4 weeks prior to the first dose of study medication (Day 1) and for the duration of the study. Cannabidiol (CBD) without THC is allowed but not on the clinical visit days except for topical applications. CBD use should be recorded as concomitant medication.
23) Prohibited prior and concomitant medications are excluded within 4 weeks prior to Screening. All allowed medications should remain stable throughout the study; for medications affecting cognition, the doses should be stable for at least 4 weeks before Screening and throughout the study, unless otherwise noted. (this is not an exhaustive list; if the permissibility of a specific medication is in question, please contact the Medical Monitor prior to randomization):
   a) Memantine in any form, combination or dosage
   b) Donepezil at 23 mg PO
   c) Antipsychotics; antipsychotics in low doses (in the judgment of the investigator) are allowed only if given for sleep disturbances, agitation and/or aggression, and only if the subject has received a stable dose for at least 3 months before Screening. If these medications are taken on a PRN basis, they should not be taken the night before any cognitive testing
   d) Tricyclic antidepressants, monoamine oxidase inhibitors, and S-ketamine; all other antidepressants are allowed only if the subject has received a stable dose for at least 3 months before Screening
   e) Anxiolytics at high doses; low doses of benzodiazepines are allowed in the judgment of the investigator, but not the night before any cognitive assessments
   f) Sedative hypnotics; Zolpidem is allowed
   g) Barbiturates (unless given in low doses for benign tremor)
   h) Nicotine therapy (including patches), varenicline (Chantix), or similar therapeutic agent
   i) Peripherally acting drugs with effects on cholinergic neurotransmission. Solifenacin is allowed if the subject has received a stable dose for at least 3 months before Screening
   j) Systemic immunosuppressants if taken in clinically immunosuppressive doses in the judgment of the investigator (note: immunosuppressant use for allergy or other inflammation, e.g., inhaled steroids, otics, opthalmologics, skin creams, and intra-articular injections are allowed)
   k) Antiepileptic medication if taken for control of seizures. Other uses, e.g., neuropathy and restless legs, are allowed
   l) Chronic intake of opioid-containing analgesics; PRN use is allowed (but not within 72 hours before any cognitive assessment)
   m) Sedating H1 antihistamines; non-sedating H1 antihistamines are allowed and preferred
   n) Systemic moderate to strong cytochrome P450 3A4 inhibitors or inducers; topical applications are allowed
24) Current enrollment in an investigational drug or device study, or have participated in another clinical trial with an investigational drug within 4 weeks of Screening, or 5 half-lives, whichever is longer, or within 6 months of Screening if an AD investigational drug.
25) The subject has received active amyloid or tau immunization (i.e., vaccination for Alzheimer's disease) at any time, or passive immunization (i.e., monoclonal antibodies for Alzheimer's disease) within 6 months of Screening. FDA approved vaccinations or monoclonal antibodies for other indications are allowed.
26) Subject has known allergy to any component of the investigational medicinal product.
27) The subject has a condition or is in a situation which, in the investigator's opinion, may put the subject at significant risk, may confound the study results, or may interfere significantly with the subject's compliance or participation in the study.

### C. Drug Product

Pre-filled syringes of ATH-1017 at 40 mg contain 1.0 mL of 40 mg/mL ATH-1017 in a solution of 10 mM sodium phosphate and 0.5% NaCl. Pre-filled syringes of ATH-1017 at 70 mg contain 1.0 mL of 70 mg/mL ATH-1017 in a solution of 10 mM sodium phosphate. Each pre-filled syringe of placebo contains 1.0 mL of a solution of 10 mM sodium phosphate and 1.1% NaCl. All solutions are adjusted to pH of approximately 7.6.

ATH-1017 and placebo are administered subcutaneously.

### D. Endpoints

The primary objectives and endpoints of this study are:

| Primary Objectives | Primary Endpoints |
|---|---|
| To evaluate the | The Global Statistical Test (GST) (O'Brien, 1984) |
| clinical efficacy | that combines the scores from cognition |
| of ATH-1017 | (Alzheimer's Disease Assessment |
| | Scale-Cognitive Subscale [ADAS-Cog₁₁]) and |
| | global impression of change (Alzheimer's |
| | Disease Cooperative Study-Clinical Global |
| | Impression of Change [ADCS-CGIC]) |
| To determine the | Analysis of adverse events (AEs), including |
| safety and | injection site AEs; changes from baseline for |
| tolerability | the following variables: vital signs, 12-lead |
| of ATH-1017 | electrocardiogram (ECG), and laboratory tests |
| | (chemistry, hematology, urinalysis); concomitant |
| | medication assessments, physical and neurological |
| | exams, Columbia-Suicide Severity Rating Scale |
| | (C-SSRS), and Geriatric Depression Scale (GDS) |

The secondary objectives and endpoints of this study are:

| Secondary Objectives | Secondary Endpoints |
|---|---|
| To evaluate the clinical efficacy of | If significance is achieved in the primary analysis in |
| ATH-1017 separately on: | GST, the secondary efficacy endpoints are tested |
| (1) cognition and | separately. |
| (2) clinical global impression of | ADAS-Cog₁₁ score: change from baseline at |
| change | Week 26 compared to placebo |
| | ADCS-CGIC: change from baseline at Week 26 |
| | compared to placebo |
| To evaluate the effect of ATH-1017 | Alzheimer's Disease Cooperative Study - Activities |
| on activities of daily living | of Daily Living, 23-item version (ADCS-ADL23) score: |
| | change from baseline at Week 26 compared to placebo |
| | (for ex-US purposes, this endpoint may function as |
| | a key secondary endpoint instead of ADCS-CGIC) |
| To determine the plasma PK profile | Day 1: post-dose anytime between 30 minutes and |
| of ATH-1017 and ATH-1001 | 120 minutes as practical* |
| | Week 12: pre-dose anytime, and post-dose anytime |
| | between 30 minutes and 120 minutes as practical* |
| | Week 26: pre-dose anytime, and post-dose anytime |
| | between 30 minutes and 120 minutes as practical* |
| | * Please record the actual time of PK sampling. |

The exploratory objectives and endpoints of this study include evaluation of behavioral changes and assessment of memory function, including:

| Exploratory Objectives | Exploratory Endpoints |
|---|---|
| To evaluate the effect | Neuropsychiatric Inventory (NPI) score: |
| of ATH-1017 on | change from baseline at Week 26 |
| behavioral changes | compared to placebo |
| To evaluate the effect | Mini Mental State Examination (MMSE) |
| of ATH 1017 on | score: change from baseline at |
| overall cognitive | Week 26 compared to placebo |
| function | |
| To evaluate the effect | Controlled Oral Word Association test |
| of ATH-1017 on | (COWAT) score: change from baseline |
| executive memory | at Week 26 compared to placebo |
| function | |
| To evaluate any effect | Zarit Burden Interview (ZBI) score: |
| of ATH-1017 on: | change from baseline at Week 26 |
| (1) caregiver burden | compared to placebo |
| (2) healthcare | Resource Utilization in Dementia lite |
| resource utilization | version (RUD-Lite ®) score: |
| (3) subject health- | change from baseline at Week |
| related quality of life | 26 compared to placebo |
| | EQ-5D-5L (Proxy 1) score: change |
| | from baseline at Week 26 |
| | compared to placebo |

### E. Screening Assessments

### 1. Mini-Mental State Examination (MMSE)

The Mini-Mental State Examination (MMSE) (Folstein, 1975) is a widely used test of overall cognitive function, assessing memory, orientation and praxis in a short series of tests. The score is from 0 to 30 with 30 being the best possible and 0 being the worst possible score. The MMSE is administered at Screening with a score of 14 to 24 inclusive for subject eligibility and at Baseline.

### 2. Clinical Dementia Rating Scale (CDR)

The Clinical Dementia Rating Scale (Hughes, 1982) is a global rating of the function of AD subjects assessed in 6 categories: memory, orientation, judgment and problem solving, community affairs, home and hobbies, and personal care. It is based on a semi-structured interview conducted with the subject and caregiver. Each category has scores from 0 (no symptoms) to 3 (severe) from which the overall CDR global score is derived. The CDR is administered at the Screening visit, with a score of 1 or 2 required for subject eligibility.

### Efficacy Variables

As specified by each assessment scale, a qualified, trained and certified rater administers questionnaires to the study subject and/or dedicated support person/caregiver. Rater training and certification (as applicable) will occur, and if necessary be repeated, in a standardized manner.

ADAS-Cog₁₁ and COWAT assessments should be done in the same time frame at all visits, in the morning.

### 1. Cognitive Variables

Alzheimer's Disease Assessment Scale—Cognitive Subscale (ADAS-Cog₁₁)

The ADAS-Cog₁₁ is designed to measure cognitive symptom changes in subjects with AD (Rosen, 1984). The standard 11 items are word recall, commands, constructional praxis, naming objects and fingers, ideational praxis, orientation, word recognition, spoken language ability, comprehension of spoken language, word-finding difficulty, and remembering test instructions. The test includes 7 performance items and 4 clinician-rated items, with a total score ranging from 0 (no impairment) to 70 (severe impairment). Therefore, higher scores indicate more severe cognitive impairment.

Due to known circadian fluctuations of cognitive capacity (Hilt, 2015), ADAS-Cog₁₁ are assessed in the morning at approximately the same time of day as the baseline assessment for all applicable visits.

ADAS-Cog₁₁ assessments are performed pre-dose at Visit 2 (Baseline/Day 1), and post-dose at approximately 1 hour (±30 minutes) at Visit 3 (Week 2), Visit 4 (Week 6), Visit 5 (Week 12), Visit 7 (Week 20), Visit 8/ET (Week 26), and Visit 9 (Safety follow-up; no dosing).

Controlled Oral Word Association Test (COWAT)

The Controlled Oral Word Association Test (COWAT) is an oral verbal fluency test in which the subject is required to make verbal associations to different letters of the alphabet by saying all the words which they can think of beginning with a given letter. Individuals are given 1 minute to name as many words as possible beginning with each of the letters. The procedure is then repeated for the remaining two letters (Benton, 1994; Strauss, 2006). The test score is the total number of different words produced for all 3 letters.

The COWAT are performed adjacent to the ADAS-Cog₁₁ assessment, i.e., pre-dose at Visit 2 (Baseline/Day 1), and post-dose at approximately 1 hour (±30 minutes) at Visit 3 (Week 2), Visit 4 (Week 6), Visit 5 (Week 12), Visit 7 (Week 20), Visit 8/ET (Week 26), and Visit 9 (Safety follow-up; no dosing).

MMSE

In addition to screening and baseline, MMSE assessments are performed post-dose at approximately 30 minutes (±15 minutes) at Visit 7 (Week 20), and Visit 8/ET (Week 26).

### 2. Disease Condition

Alzheimer's Disease Cooperative Study Clinical Global Impression of Change (ADCS-CGIC)

The Alzheimer's Disease Cooperative Study Clinical Global Impression of Change (ADCS-CGIC) scale is a 7-point scale that requires the clinician to assess how much the subject's illness has improved or worsened relative to a baseline state at the beginning of the intervention and rated as: 1, markedly improved; 2, moderately improved; 3, minimally improved; 4, no change; 5, minimally worse; 6, moderately worse; or 7, markedly worse. The ADCS-CGIC consists of 3 parts: a guided baseline interview administered to the subject and caregiver/support person, a follow-up interview administered to the subject and caregiver/support person, and a clinician's rating review (Schneider, 1997). At study start, using the ADCS-CGIC baseline form as a guideline, the ADCS-CGIC rater obtains an integral clinical impression of the subject's status, can apply any formal testing at his/her discretion, and take personal notes regarding the subject's condition; these serve as a reference for future change ratings. The ADCS-CGIC are administered by an experienced clinician who remains independent of the subject's safety, cognitive and functional outcomes, and are trained and certified for this study. The ADCS-CGIC rater ideally remains the same individual for all ADCS-CGIC ratings.

ADCS-CGIC assessments are performed adjacent to ADAS-Cog₁₁ and COWAT assessments pre-dose at Visit 2 (Baseline/Day 1), post-dose at Visit 5 (Week 12), and post-dose at Visit 8/ET (Week 26).

Alzheimer's Disease Cooperative Study—Activities of Daily Living, 23-Item Version (ADCS-ADL23)

The ADCS-ADL23 (Galasko, 1997) is a 23-item assessment of functional impairment in terms of activities of daily living administered to the support person/caregiver. It comprises 23 questions about the subject's involvement and level of performance across items representing daily living. The questions range from basic to instrumental activities of daily living. Each item is rated from the highest level of independent performance to complete loss. The total score range is from 0 to 78, with lower scores indicating greater functional impairment. ADCS-ADL23 assessments are performed pre-dose at Visit 2 (Baseline/Day 1), post-dose at Visit 5 (Week 12), and post-dose at Visit 8/ET (Week 26).

Neuropsychiatric Inventory (NPI)

The NPI is an interview-based rating scale of psychiatric and behavioral disturbances associated with dementia (Cummings, 1994). The support person/caregiver is interviewed by the qualified NPI rater about the presence or absence of 12 symptoms, including delusions, hallucinations, agitation/aggression, depression/dysphoria, anxiety, elation/euphoria, apathy/indifference, disinhibition, irritability/lability, aberrant motor behavior, sleep and nighttime behavior disorders, and appetite and eating disorders. For those symptoms present, the support person/caregiver rates the frequency, severity, and distress of each symptom. A total score and a caregiver distress score are calculated. A higher score indicates more severe psychopathology.

NPI assessments are performed pre-dose at Visit 2 (Baseline/Day 1), post-dose at Visit 5 (Week 12), and post-dose at Visit 8/ET (Week 26).

### 3. Health-Related Quality of Life

EuroQol Group 5-Dimension 5 Level Questionnaire (EQ-5D-5L)

The EQ-5D-5L Health Questionnaire (The EuroQol Group, 1990) is a standardized instrument that offers a simple method for obtaining a self-rating of current health status on a vertical visual analog scale (VAS) of 0-100 (Kind, 1996). Higher scores on the VAS indicate a better health state.

The EQ-5D-5L Health Questionnaire (Proxy version 1) are completed pre-dose at Visit 2 (Baseline/Day 1) and post-dose at Visit 8/ET (Week 26).

### 4. Caregiver Burden/Resource Utilization/Pharmacoeconomic Variables

Zarit Burden Interview (ZBI)

The ZBI is a 22-item questionnaire specifically designed to reflect the stress experienced by caregivers of subjects with dementia (Zarit, 1980). The support person/caregiver answers questions about the impact of the subject's disabilities on their life. The score for each individual item ranges from 0 to 4 (never, rarely, sometimes, quite frequently, and nearly always) with a total score range from 0 to 88; a higher score reflects a higher degree of caregiver perceived burden or stress.

ZBI assessments are performed pre-dose at Visit 2 (Baseline/Day 1) and post-dose at Visit 8/ET (Week 26).

Resource Utilization in Dementia (RUD-Lite®)

The RUD instrument is designed to assess caregiver burden and provide pharmacoeconomic data related to AD (Wimo, 1998). The RUD-Lite®, used in this study, includes baseline and follow-up questions. Caregiver-related questions include a description of the caregiver (demographic information) and time spent caring for the subject and changes in work status. Subject-related questions include residential status and health care resource utilization. Note that the caregiver-related use of health resources included in the RUD is not part of the RUD-Lite®.

RUID-Lite® assessments are performed pre-dose at Visit 2 (Baseline/Day 1) and post-dose at Visit 8/ET (Week 26).

### F. Schedule and Order of Assessments

The study consists of up to 28 days of Screening (Day −28 through Day −1) followed by 26 weeks of double-blind treatment and a 4-week safety follow-up. Note: if 28 days is not sufficient to complete the screening period, the possibility of an extension can be discussed with the Medical Monitor.

For clinical outcome assessment evaluating subject's cognitive condition, the general order should be followed:

| | |
|---|---|
| (1) | MMSE |
| (2) | ADAS-Cog11 |
| (3) | COWAT |
| (4) | ADCS-CGIC |

At Baseline/Day 1 (Visit 2), MMSE should be done first before all other assessments pre-dose.

MMSE assessments are performed first before all other clinical outcome assessments post-dose at approximately 30 minutes (±15 minutes) at Week 20 (Visit 7), and Week 26 (Visit 8).

ADAS-Cog₁₁ and COWAT assessments shall occur at clinic visits in the morning at approximately the same time they were performed during the initial Baseline/Day 1 assessment. ADAS-Cog₁₁ and COWAT assessments are performed pre-dose at Baseline/Day 1 (Visit 2), and post-dose at approximately 1 hour (±30 minutes) at Week 2 (Visit 3), Week 6 (Visit 4), Week 12 (Visit 5), Week 20 (Visit 7), and Week 26 (Visit 8); and additionally at Safety follow up (visit 9; no dosing).

ADCS-CGIC assessments are organized at adjacent times to the individual ADAS-Cog₁₁ and COWAT assessment times at Baseline/Day 1 (Visit 2), and post-dose at Week 12 (Visit 5), and Week 26 (Visit 8).

PK plasma samples are collected at post-dose at Baseline/Day 1 (Visit 2); pre-dose and post-dose at Week 12 (Visit 5) and Week 26 (Visit 8). The pre-dose PK sample is collected anytime before dosing. The post-dose PK sample is collected anytime between 30 minutes and 120 minutes after dosing as practical. The actual time of dosing and of PK sampling are recorded.

The order of assessments for all other endpoints is flexible.

### G. Statistical Methods

A statistical analysis plan (SAP) are issued, providing detailed methods for the analyses outlined below.

### Populations for Analysis

### MITT Population

The modified intent-to-treat (mITT) population includes all randomized subjects who took at least one dose of the study medication and who completed both an ADAS-Cog₁₁ and ADCS-CGIC assessment during at least one post-baseline visit. Subjects are analyzed according to the dose they were randomized to.

### Per Protocol Population

The per protocol population includes all mITT subjects who took the assigned medication during the 26 weeks of treatment, completed both an ADAS-Cog11 and ADCS-CGIC assessment during at least one post-baseline visit, and did not have any major protocol deviations. Subjects are analyzed based on actual treatment received.

### Safety Population

The safety population includes all randomized subjects who received at least one dose of the study medication. Subjects are analyzed based on actual treatment received.

### General Considerations

Descriptive statistics for continuous variables include number of subjects (n), arithmetic mean, standard deviation, median, minimum, maximum and first and third quartile limits unless otherwise noted. Frequency and percentage are calculated for categorical variables.

Change from baseline is calculated by subtracting the baseline score from the observed value at any subsequent visit. For safety summaries, the last pre-randomization measurement is defined as the baseline value. For efficacy measures baseline is defined as the last pre-randomization measurement.

Percentages are based on the number of subjects in each treatment group in the given population for AE summary tables, and additionally overall for medical history, prior and concomitant medications. For all other tables, percentages are based on the number of subjects with non-missing data in each treatment group and overall for the given population.

### Efficacy Analyses

### Primary Efficacy Analysis—GST

The primary efficacy hypothesis is that treatment with ATH-1017 results in a statistically significant reduction in change from baseline in the Global Statistical Test score (GST; O'Brien, 1984) (combining the ADAS-Cog11 score and the ADCS-CGIC score) relative to the placebo group at Week 26 in the mITT population. The primary analysis tests the statistical hypothesis of no difference between placebo and each of the 2 treatment groups (40 mg ATH-1017 and 70 mg ATH-1017).

The primary analysis uses a mixed model for repeated measures (MMRM) to compare the estimated change from baseline between active treatment and placebo in the GST score. This analysis assesses whether or not there is a difference in estimated GST values between treatment groups and placebo at 26 weeks using least squares means estimates from the MMRM model, and includes terms for baseline, baseline by time interaction, and baseline by time by treatment interaction in the model. Additional terms are included for ApoE genotype, site, with smaller sites grouped, age, and MMSE scores. Least squares means and standard errors are estimated from the MMRM model at Week 26. Further details relating to the primary analysis are described in the SAP.

### Secondary Efficacy Analysis—ADAS-Cog11 and ADCS-CGIC

The separate key secondary efficacy endpoints of ADAS-Cog11 and ADCS-CGIC are analyzed using the same MMRM model that was used for analysis of the primary endpoint. Since the ADCS-CGIC is already a comparison to baseline, the absolute score is used for analysis instead of a calculated change from baseline.

A gatekeeper strategy is used to preserve the family-wise alpha error at the 2-sided level of 0.05 between co-primary endpoints and the multiple doses. If significance is achieved for a given dose in the primary analysis in GST after the Hochberg procedure at 0.05, then testing can proceed for the secondary efficacy endpoints of ADAS-Cog11 and ADCS-CGIC in that dose, which is also co-primary at 0.05. If both doses achieve significance in the primary analysis, the doses are analyzed in sequence, so that the second dose in the sequence can only be declared significant if the first dose in the sequence achieves significance. The sequence order are determined by the significance level achieved in the primary analysis, i.e. the dose with the lower p-value when testing the GST are first in the sequence for the secondary analysis.

If both ADAS-Cog11 and ADCS-CGIC are significant at an alpha level of 0.05 for the first dose analyzed, the study is considered a pivotal study with co-primary endpoints. The other dose is then compared to placebo for both co-primary endpoints using the same strategy as used for the initial dose tested. In addition, an exploratory analysis comparing a pooled active treatment group to placebo is performed.

Additional endpoints are tested in a hierarchical (gated) approach in the following order: ADCS-ADL23, NPI, MMSE, COWAT, EQ-5D-5L, ZBI, and RUD-Lite®.

For ex-US purposes, the activities of daily living endpoint, change from baseline at Week 26 compared to placebo for ADCS-ADL23, may function as a key secondary endpoint instead of ADCS-CGIC.

### Subgroup Analyses

Subgroup analyses (e.g., gender, age, MMSE score, ApoE genotype) are performed in the mITT population.

### H. Results

Treatment with ATH-1017 achieves one or more of the primary, secondary, or exploratory endpoints, while having acceptable safety and tolerability.

### Example 2: Translational Study Evaluating ATH-1017 in Subjects with Mild to Moderate Alzheimer's Disease

### A. Overview of Study Design

In human clinical studies of ATH-1017, single SC administration of 2, 6, 20, 40, 60, and 90 mg in healthy young subjects, and multiple administration of 20, 40, 60, and 80 mg (SC, OD, over 9 consecutive days) in healthy elderly subjects, and 40 mg (SC, OD, over 9 consecutive days) in AD subjects were safe and well tolerated. Injection site reactions included pain, pruritus, and/or erythema, were mild in nature, and resolved without specific therapy. A potential risk for hepatotoxicity identified in nonclinical studies has not been observed in human studies but is closely monitored in this study. To date, no CNS-specific adverse events have been observed in humans.

This is a Phase 2 multicenter, randomized, double-blind, placebo-controlled, parallel-group, dose-ranging study comparing ATH-1017 40 mg/day and ATH-1017 70 mg/day with placebo in subjects with a clinical diagnosis of mild to moderate AD, diagnosed on a ‘probable’ level according to McKhann, 2011. The study is conducted at a total of approximately 14 centers in Australia and the US. Subjects and their caregivers are required to sign an informed consent form (ICF) and are evaluated against the inclusion/exclusion criteria during a screening period; all eligible subjects are tested for ApoE genotype. Subjects who meet all inclusion/exclusion criteria undergo baseline EEG assessments (ERP P300 and qEEG) at 2 separate baseline visits. At the first baseline visit (Visit 2a, Pre-baseline, Day −5 to Day −3), Mini-Mental State Examination (MMSE) is performed first, followed by EEG assessments (ERP P300 and qEEG) at 2 separate timepoints approximately 2 hours apart (no dosing). EEG data should be uploaded for quality check immediately after the completion of the Pre-baseline visit (Visit 2a). At the second baseline visit (Visit 2b, Baseline, Day 1), no more than 6 days after the Pre-baseline visit, subjects are randomized in a ratio of 1:1:1 to 3 parallel arms, either to active treatment (ATH-1017 40 mg/day or ATH-1017 70 mg/day) or placebo. During randomization, subjects are stratified by screening MMSE severity: mild (MMSE: 20-24) versus moderate (MMSE: 14-19). At this Baseline visit (Visit 2b), subjects undergo pre-dose baseline and post-dose EEG assessments (ERP P300 and qEEG).

Study drugs are administered by SC injection OD preferably by during daytime. Do not take more than one dose within 8 hours. The first SC injection of study drug is performed at site under supervision. The subject should withhold study drug administration on the day of subsequent clinic visits; study drug administration is done on site under supervision of site staff at these visits. Each subject is required to have a primary caregiver willing to accept responsibility for supervising or, if required, administering study drug, and assessing the condition of the subject throughout the study in accordance with all protocol requirements. During the double-blind treatment period, clinic visits take place on Day 1 and thereafter at Weeks 2, 6, 12, 16, 20, and 26, with a safety follow-up visit scheduled 4 weeks after completion of the double-blind period at Week 30. Subjects undergo EEG assessments (ERP P300 and qEEG) at each post-baseline clinic visit (pre- and post-dose timepoints) through Week 26, plus the safety follow-up visit at Week 30. On Day 1, after completion of the first dose, subjects remain on-site 2 hours for post-treatment safety observation. As marked circadian fluctuations of cognitive performance have been observed in AD (Hilt, 2015), ADAS-Cog₁₁ and COWAT assessments shall occur at clinic visits in the morning at approximately the same time they were performed during the initial Baseline assessment. Similarly, ADCS-CGIC assessments are organized at adjacent times to the individual EEG assessment times. Subjects may live at home, in a senior residential setting, or an institutional setting without the need for continuous nursing care, and should not be likely to experience a change in living conditions (e.g., institutionalization, moving to a different city, etc.), or change in primary caregiver, during participation in the trial period. The end of the study is defined as the date of the safety follow-up visit, Visit 9/Week 30. Subjects who terminate prior to Visit 8 are to complete same assessments as Visit 8/early termination (ET).

An independent Data Safety Monitoring Board conducts periodic review and assessments of unblinded safety data (AEs, labs, ECG, etc.) throughout the study to ensure the safety of study subjects.

Blood draws take place at scheduled clinic visits (Day 1, Week 12 and Week 26) for analysis of plasma concentrations of ATH-1017 and ATH-1001.

A 26-week open-label extension is offered at participating sites.

### B. Subject Population

The study randomizes approximately 75 subjects in a 1:1:1 ratio to ATH-1017 40 mg, ATH-1017 70 mg, and placebo groups subjects in order to include a total of approximately 60 evaluable subjects in the analysis of the primary endpoint. Inclusion Criteria

Subjects must meet all of the following inclusion criteria to participate in the study.
1) Age 55 to 85 years, inclusive at the time of signing the informed consent.
2) Mild-to-moderate AD dementia subjects.
   a) MMSE score 14 to 24 inclusive at Screening
   b) Clinical Dementia Rating (CDR) Scale global score of 1 or 2 at Screening
3) Clinical diagnosis of dementia, due probably to AD, by Revised National Institute on Aging-Alzheimer's Association criteria (McKhann, 2011):
   a) Magnetic resonance imaging (MM) or computerized tomography (CT) scan (for subjects with non-MRI-safe cardiac pacemaker, or other relevant medical reason, with Medical Monitor approval) performed within 12 months before Screening, with findings consistent with the diagnosis of dementia due to AD without any other significant comorbid central nervous system pathologies. If such scan is unavailable or older than 12 months, it should be repeated to ascertain the diagnosis before randomization.
   b) Documented clinical decline within 12 months before Screening and onset of symptoms at least 12 months before Screening (preferably subject medical records; caregiver reports with examples are acceptable).
4) Formal education of 8 or more years; exceptions may be made for subjects with less than 8 years of education at the discretion of the investigator.
5) Body mass index (BMI) of ≥18 and ≤35 kg/m2 at Screening; subjects with BMI outside the allowed BMI range but ≥16 and ≤37 kg/m2 may enroll only with prior agreement of the Sponsor.
6) Male subjects and their partners must agree to use a double-barrier method of contraception during the study, including the follow-up period, unless the partner is not of childbearing potential. Only female subjects of non-childbearing potential (i.e., permanently sterilized, postmenopausal) are eligible for participation.
7) Reliable and capable support person/caregiver, who is willing to accept responsibility for supervising the treatment or, if required, administering study drug and assessing the condition of the subject throughout the study in accordance with all protocol requirements. The support person/caregiver must see the subject at least once-daily for dose administration and/or observation and have approximately 4 to 6 hours daytime contact with the subject for at least 4 days/week.
8) Treatment-free or receiving stable acetylcholinesterase inhibitor (AChEI) treatment, defined as:
   a) Treatment-naïve, OR
b) Concomitant therapy with AChEI is allowed as long as the dose has been stable for 3 months prior to Screening (cf. EC #25b) and no changes are planned during the study, OR
c) Subjects who received an AChEI in the past and discontinued at least 4 weeks prior to Screening.
9) Subject capable of giving signed informed consent, which includes compliance with the requirements and restrictions listed in the ICF and in this protocol. If the subject is incapable of giving informed consent in the judgment of the investigator then consent may be provided by a legally acceptable representative.
10) Written informed consent from a) the subject or legally acceptable representative and b) caregiver/support person has been obtained prior to any study-related procedures, including prior to initiating procedures to evaluate eligibility for the study.
11) Written documentation has been obtained in accordance with the relevant country and local privacy requirements, where applicable (e.g., Written Authorization for Use and Release of Health and Research Study Information).
12) Subjects and caregivers/support persons are able, as assessed by the investigator, and willing to follow study instructions and likely to complete all required study visits.
13) Subjects must be in generally good health as assessed by the investigator from medical history and physical/neurological examination, vital signs, ECG, and standard laboratory tests.

### Exclusion Criteria

Subjects who meet any of the following criteria are excluded from the study:
1) History of significant neurologic disease, other than AD, that may affect cognition, or concurrent with the onset of dementia.
2) History of unexplained loss of consciousness, and epileptic fits (unless febrile). 3) Subject has atypical variant presentation of AD, if known from medical history, particularly non-amnestic AD.
4) History of brain MRI scan indicative of any other significant abnormality, including but not limited to multiple (>10) microhemorrhages, severe white matter hyperintensities, history or evidence of a single prior hemorrhage >1 cm³, multiple (>3) lacunar infarcts or evidence of a single prior infarct >1 cm³, evidence of a cerebral contusion, encephalomalacia, aneurysms, vascular malformations, subdural hematoma, or space-occupying lesions (e.g., brain tumors). If a known meningioma has been stable for >1 year and the subject has no history of any type of convulsions, this can be allowable after consultation with the Medical Monitor. Note: a new MM scan is required if the scan was performed >12 months prior to Screening; a repeat MRI scan is required if there have been intervening changes to the subject's clinical presentation in the past 12 months. CT scan is acceptable for subjects fitted with non-MRI-safe cardiac pacemaker or other relevant medical reason, with Medical Monitor approval.
5) Inability to hear or differentiate the two different tones necessary for auditory ERP P300 assessment, using the centrally provided EEG equipment; hearing aid must be removed during the screening hearing test and during EEG recordings.
6) Diagnosis with current symptoms of severe major depressive disorder even without psychotic features. Any subject with formalized delusions or hallucinations is excluded.
7) Geriatric Depression Scale (GDS) score (15-item scale)>7 at Screening. In discussion with the Medical Monitor, subjects with a GDS score between 8 and 10 inclusive can be considered for study participation if the increased score is driven by specific domains related to the pandemic and its restrictions, rather than by major depression.
8) Significant suicide risk as defined by suicidal ideation based on the Columbia-Suicide Severity Rating Scale (C-SSRS) within the last 12 months, at Screening and on Day 1 (i.e., a ‘yes’ response to Question 4 or 5, or any specific behaviours).
9) History within 2 years of Screening, or current diagnosis of psychosis (American Psychiatric Association, 2000) or moderate substance abuse disorder (according to the Diagnostic and Statistical Manual of Mental Disorders, Fifth Edition).
10) Untreated conditions, including vitamin B12 or folate deficiency, hypothyroidism, diabetes mellitus, hypo- or hypertension, if clinically relevant in the judgment of the investigator. If treated, must be stably treated and symptom-free for at least 6 months before Screening.
11) Abnormal serum electrolytes (potassium, sodium, magnesium) of clinical significance. If treated, must be stably treated for at least 30 days before Screening.
12) Active, acute, or chronic infectious disease of any type.
13) Myocardial infarction or unstable angina within the last 6 months or history of more than one myocardial infarction within 5 years before Screening.
14) Clinically significant (in the judgment of the investigator) cardiac arrhythmia (including atrial fibrillation), cardiomyopathy, or cardiac conduction defect (note: pacemaker is acceptable).
15) Subject has either hypertension (supine diastolic blood pressure >95 mmHg), or symptomatic hypotension in the judgment of the investigator.
16) Clinically significant ECG abnormality at Screening, including but not limited to a confirmed corrected QT interval using Fridericia's formula (QTcF) value ≥450 msec for males and ≥470 msec for females. For QTcF readings that are borderline, or difficult to interpret due to e.g., presence of a Branch Bundle Block, or in those where the T and U waves are superimposed or connected, a manual, local reading using the same ECG device should be considered to determine eligibility, in discussion with the Medical Monitor. In subjects with a QRS value >120 msec, those with a QTcF value <500 msec may be eligible following discussion with the Medical Monitor.
17) History of or positive results of serology screening for hepatitis B (hepatitis B surface antigen [HBsAg]), hepatitis C (anti-hepatitis C virus [HCV] antibodies) or human immunodeficiency virus (HIV) (antibodies type 1 and 2).
18) Renal insufficiency (serum creatinine >2.0 mg/dL). 19) Hepatic impairment with alanine aminotransferase (ALT) or aspartate aminotransferase (AST)>2 times the upper limit of normal, or Child-Pugh class B and C.
20) Malignant tumor within 3 years before Screening, except for the following conditions that are stable in the judgement of the Investigator.
   a) Adequately treated squamous and basal cell carcinoma, or squamous and basal cell carcinoma in situ
   b) Prostate carcinoma in situ
21) Clinically significant (in the judgment of the investigator) unintentional weight loss within 12 months of Screening.
22) The consumption of grapefruit or grapefruit-containing products is prohibited beginning 7 days prior to the first dose of study medication (Day 1) and during the study.
23) Food supplements and nutraceuticals with potential effects on cognition, such as Axona and mediumchain triglyceride (MCT), are prohibited beginning 7 days prior to the first dose of study medication (Day 1) and for the duration of the study.
24) Tetrahydrocannabinol (THC) is prohibited beginning 4 weeks prior to the first dose of study medication (Day 1) and for the duration of the study. Cannabidiol (CBD) without THC is allowed but not on the clinical visit days except for topical applications. CBD use should be recorded as concomitant medication.
25) Prohibited prior and concomitant medications are excluded within 4 weeks prior to Screening. All allowed medications should remain stable throughout the study; for medications affecting cognition, the doses should be stable for at least 4 weeks before Screening and throughout the study, unless otherwise noted. (this is not an exhaustive list; if the permissibility of a specific medication is in question, please contact the Medical Monitor prior to randomization):
   a) Memantine in any form, combination or dosage
   b) Donepezil at 23 mg PO
   c) Antipsychotics; antipsychotics in low doses (in the judgment of the investigator) are allowed only if given for sleep disturbances, agitation and/or aggression, and only if the subject has received a stable dose for at least 3 months before Screening. If these medications are taken on a PRN basis, they should not be taken the night before any cognitive testing.
   d) Tricyclic antidepressants, monoamine oxidase inhibitors, and Sketamine; all other antidepressants are allowed only if the subject has received a stable dose for at least 3 months before Screening
   e) Anxiolytics at high doses; low doses of benzodiazepines are allowed in the judgment of the investigator, but not the night before any cognitive assessments.
   f) Sedative hypnotics; Zolpidem is allowed
   g) Barbiturates (unless given in low doses for benign tremor)
   h) Nicotine therapy (including patches), varenicline (Chantix), or similar therapeutic agent
   i) Peripherally acting drugs with effects on cholinergic neurotransmission. Solifenacin is allowed if the subject has received a stable dose for at least 3 months before Screening.
   j) Systemic immunosuppressants if taken in clinically immunosuppressive doses in the judgment of the investigator (note: immunosuppressant use for allergy or other inflammation, e.g., inhaled steroids, otics, opthalmologics, skin creams, and intra-articular injections are allowed)
   k) Antiepileptic medications
   l) Chronic intake of opioid-containing analgesics; PRN use is allowed (but not within 72 hours before any cognitive assessment)
   m) Sedating H₁ antihistamines; non-sedating H1 antihistamines are allowed and preferred
   n) Systemic moderate to strong cytochrome P450 3A4 (CYP3A4) inhibitors or inducers; topical applications are allowed
26) Current enrollment in an investigational drug or device study, or have participated in another clinical trial with an investigational drug within 4 weeks of Screening, or 5 half-lives, whichever is longer, or within 6 months of Screening if an AD investigational drug.
27) The subject has received active amyloid or tau immunization at any time (i.e., vaccination for Alzheimer's disease), or passive immunization (i.e., monoclonal antibodies for Alzheimer's disease) within 6 months of Screening. FDA approved vaccinations or monoclonal antibodies for other indications are allowed.
28) Subject has known allergy to any component of the ATH-1017.
29) The subject has a condition or is in a situation which, in the investigator's opinion, may put the subject at significant risk, may confound the study results, or may interfere significantly with the subject's compliance or participation in the study.

The initial study population included 77 subjects with mild-to-moderate Alzheimer's Disease dementia. The subjects ranged in age from 55-85 years old, with a CDR Scale global core of 1 or 2, and an MMSE score of 14-24. Baseline demographics are shown in the table below.

| | Overall^{b} |
|---|---|
| | (N = 77) |
| Age at informed consent (years); mean (SD) | 71.4 ± 7.3 |
| Body mass index (kg/m²), mean (SD) | 25.4 ± 3.7 |
| Sex, n (%) | |
| Female | 39 (50.6%) |
| Male | 38 (49.4%) |
| Years of education, mean (SD) | 14.9 ± 2.8 |
| Baseline MMSE, mean (SD) | 19.3 ± 2.7 |
| Currently taking an AChEI, n (%) | 46 (59.7%) |
| ERP P300 Latency^{b} | 381 ± 4.1 |

^{b}= preliminary population of 77 patients

### C. Drug Product

Pre-filled syringes of ATH-1017 at 40 mg contain 1.0 mL of 40 mg/mL ATH-1017 in a solution of 10 mM sodium phosphate and 0.5% NaCl. Pre-filled syringes of ATH-1017 at 70 mg contain 1.0 mL of 70 mg/mL ATH-1017 in a solution of 10 mM sodium phosphate. Each pre-filled syringe of placebo contains 1.0 mL of a solution of 10 mM sodium phosphate and 1.1% NaCl. All solutions are adjusted to pH of approximately 7.6.

ATH-1017 and Placebo are Administered Subcutaneously.

### D. Endpoints

The primary objectives and endpoints of this study are:

| Primary Objectives | Primary Endpoints |
|---|---|
| To evaluate the effects of | ERP P300 latency: changes at Weeks 2, 6, 12, 16, 20, |
| ATH-1017 on event-related | and 26 compared to placebo |
| potential (ERP) P300 latency | |
| To determine the safety and | Analysis of adverse events (AEs), including injection site |
| tolerability of ATH-1017 | AEs; changes from baseline for the following variables: |
| | vital signs, 12-lead electrocardiogram (ECG), and laboratory |
| | tests (chemistry, hematology, urinalysis); concomitant |
| | medication assessments, physical and neurological exams, |
| | Columbia-Suicide Severity Rating Scale (C-SSRS), and |
| | Geriatric Depression Scale (GDS) |

The secondary objectives and endpoints of this study are:

| Secondary Objectives | Secondary Endpoints |
|---|---|
| To evaluate the correlation of | Correlation of ERP P300 latency and |
| ERP P300 latency and cognition | cognition/executive memory function: changes at |
| measured by Alzheimer's Disease | Weeks 2, 6, 12, 20, and 26 compared to placebo |
| Assessment Scale-Cognitive | |
| Subscale (ADAS-Cog₁₁) and/or | |
| executive memory function | |
| measured by Controlled Oral | |
| Word Association test (COWAT) | |
| To evaluate the clinical | The Global Statistical Test (GST) (O'Brien, 1984) that |
| efficacy of ATH-1017 | combines the scores from cognition (Alzheimer's |
| | Disease Assessment Scale-Cognitive Subscale |
| | [ADAS-Cog₁₁]) and global impression of change |
| | (Alzheimer's Disease Cooperative Study-Clinical |
| | Global Impression of Change [ADCS-CGIC]) |
| To evaluate the effect of | ADAS-Cog₁₁ score: change from baseline at Week 26 |
| ATH-1017 on cognition | compared to placebo |
| To evaluate the effect of | ADCS-CGIC score: change from baseline at Week 26 |
| ATH-1017 on clinical global | compared to placebo |
| impression of change | |
| To evaluate the effect of | Alzheimer's Disease Cooperative Study - Activities of |
| ATH-1017 on activities of | Daily Living, 23-item version (ADCS-ADL23) score: |
| daily living | change from baseline at Week 26 compared to placebo |
| To determine the plasma PK | Day 1: post-dose anytime between 30 minutes and |
| profile of ATH-1017 and | 120 minutes as practical* |
| ATH-1001 | Week 12: pre-dose anytime, and post-dose anytime |
| | between 30 minutes and 120 minutes as practical* |
| | Week 26: pre-dose anytime, and post-dose anytime |
| | between 30 minutes and 120 minutes as practical* |
| | * Please record the actual time of PK sampling. |

The exploratory objectives and endpoints of this study are:

| Exploratory Objectives | Exploratory Endpoints |
|---|---|
| To further evaluate the | Persistence of change in ERP P300 |
| effects of ATH-1017 | latency at Week 30 compared to placebo |
| on ERP P300 latency | |
| To evaluate the effect | COWAT score: change from baseline at |
| of ATH-1017 on | Week 26 compared to placebo |
| executive memory | |
| function | |
| To evaluate the effects | Spectral analysis of absolute and/or |
| of ATH-1017 on | relative amplitude in 0.5 Hz bands from |
| quantitative EEG | 0.5 to 58.0 Hz, changes at Weeks 2, 6, |
| (qEEG) | 12, 16, 20, and 26 compared to placebo |
| To evaluate the effect | Neuropsychiatric Inventory (NPI) score: |
| of ATH-1017 on | change from baseline at Week 26 |
| behavioral changes | compared to placebo |
| To evaluate any effect | Zarit Burden Interview (ZBI) score: |
| of ATH-1017 on: | change from baseline at Week 26 |
| (1) caregiver burden | compared to placebo |
| (2) healthcare | Resource Utilization in Dementia lite |
| resource utilization | version (RUD-Lite ®) score: change |
| (3) subject health- | from baseline at Week 26 compared |
| | to placebo |
| related quality of life | EQ-5D-5L (Proxy 1) score: change from |
| | baseline at Week 26 compared to placebo |

### E. Screening Assessments

### 1. Mini-Mental State Examination (MMZE)

The Mini-Mental State Examination (MMSE) (Folstein, 1975) is a widely used test of overall cognitive function, assessing memory, orientation and praxis in a short series of tests. The score is from 0 to 30 with 30 being the best possible and 0 being the worst possible score. The MMSE is administered at Screening with a score of 14 to 24 inclusive for subject eligibility and at the Pre-baseline visit (Visit 2a, Day −5 to Day −3). At Pre-baseline visit (Visit 2a, Day −5 to Day −3), MMSE should be done first before all other assessments.

### 2. Clinical Dementia Rating Scale (CDR)

The Clinical Dementia Rating Scale (Hughes, 1982) is a global rating of the function of AD subjects assessed in 6 categories: memory, orientation, judgment and problem solving, community affairs, home and hobbies, and personal care. It is based on a semi-structured interview conducted with the subject and caregiver. Each category has scores from 0 (no symptoms) to 3 (severe) from which the overall CDR global score is derived. The CDR is administered at the Screening visit with a score of 1 or 2 required for subject eligibility.

### 3. Audio Screening

A brief hearing test is performed at the Screening visit for the purpose of documenting that subjects have adequate hearing to participate in the auditory ERP P300 procedure, i.e., ability to hear and differentiate the two different tones, using the centrally provided EEG equipment; hearing aid must be removed during the screening hearing test and during EEG recordings.

### Pharmacodynamic Variables

Pharmacodynamic variables consist of EEG assessments (ERP P300 and qEEG) performed over approximately 20 minutes, with ERP P300 performed prior to qEEG.

At Pre-baseline visit (Visit 2a, Day −5 to Day −3, no dosing), EEG assessments (ERP P300 and qEEG) are performed twice, approximately 2 hours apart. EEG data should be uploaded for quality check immediately after the completion of the Pre-baseline visit (Visit 2a).

At Baseline/Day 1 (Visit 2b), EEG assessments (ERP P300 and qEEG) are performed at pre-dose following the completion of baseline assessments of ADAS-Cog₁₁ and COWAT, and before the ADCS-CGIC assessment, up to 1.5 hour before dose in clinic. EEG is assessed post-dose at approximately 2 (±1) hours after ATH-1017 dosing.

At Visits 3, 4, 5, 6, 7, and 8, EEG assessments (ERP P300 and qEEG) are performed at pre-dose up to 1 hour before dose in clinic. EEG is assessed post-dose following the completion of ADAS-Cog₁₁ and COWAT assessments, and before the ADCS-CGIC assessment, at approximately 2 (±1) hours after ATH-1017 dosing.

At safety follow up (Visit 9, no dosing), EEG assessments (ERP P300 and qEEG) are performed following the completion of ADAS-Cog₁₁ and COWAT.

### 1. ERP P300

ERP P300 is a method of recording brain activity elicited by external stimuli, e.g., an oddball auditory stimulus, and is a well-established functional biomarker, particularly of working memory access (Ally, 2006). ERP P300 is characterized by a stereotyped series of voltage deflections occurring after the respective odd tone to be counted, with early features (<100 msec) corresponding to unconscious sensory transmission (auditory cortex, N100), and later features produced by cognitive processing in the ventral attentional network, i.e., P300, referring to the large positive deflection at roughly 300 msec in healthy adults (young or elderly). The P300 latency is sensitive to detecting reduced synaptic transmission related to cognitive decline in AD patients and other dementias (Olichney, 2011).

To assess the P300 wave (latency and amplitude), the subject has to perform a task related to auditory stimuli. The stimulus consists of an oddball paradigm with 2 sound stimuli. Stimuli are presented through headphones and auditory stimulation for P300 is assessed in a recording lasting up to 10 minutes.

2. qEEG

Modulation of EEG signatures by pharmacological agents indicates CNS penetration and suggests target engagement. qEEG has been shown to serve as a non-invasive translational biomarker from preclinical to clinical studies (Leiser, 2011).

The qEEG procedure is conducted with the subject in a semi-sitting position (resting condition) in a quiet environment. For each timepoint, spontaneous EEGs are recorded with 5 minutes ‘eyes closed’ followed by 5 minutes ‘eyes open’. Spectral analysis of absolute and/or relative amplitude in 0.5 Hz bands from 0.5 to 58.0 Hz is performed. Results are presented graphically using brain map representations.

### Other Variables

As specified by each assessment scale, a qualified, trained and certified rater administers questionnaires to the study subject and/or dedicated support person/caregiver. Rater training and certification (as applicable) occurs, and if necessary is repeated, in a standardized manner.

ADAS-Cog₁₁ and COWAT assessments shall occur at clinic visits in the morning at approximately the same time they were performed during the initial Baseline/Day 1 assessment.

### 1. Cognitive Variables

Alzheimer's Disease Assessment Scale—Cognitive Subscale (ADAS-Cog₁₁)

The ADAS-Cog₁₁ is designed to measure cognitive symptom changes in subjects with AD (Rosen, 1984). The standard 11 items are word recall, commands, constructional praxis, naming objects and fingers, ideational praxis, orientation, word recognition, spoken language ability, comprehension of spoken language, word-finding difficulty, and remembering test instructions. The test includes 7 performance items and 4 clinician-rated items, with a total score ranging from 0 (no impairment) to 70 (severe impairment). Therefore, higher scores indicate more severe cognitive impairment.

Due to known circadian fluctuations of cognitive capacity (Hilt, 2015), ADAS-Cog₁₁ is assessed in the morning at approximately the same time of day as the baseline assessment for all applicable visits.

ADAS-Cog₁₁ assessments are performed pre-dose at Visit 2b (Baseline/Day 1), and post-dose at approximately 1 hour (±30 minutes) at Visit 3 (Week 2), Visit 4 (Week 6), Visit 5 (Week 12), Visit 7 (Week 20), Visit 8/ET (Week 26), and Visit 9 (Safety follow-up; no dosing.

Controlled Word Association Test (COWAT)

The Controlled Oral Word Association Test (COWAT) is an oral verbal fluency test in which the subject is required to make verbal associations to different letters of the alphabet by saying all the words which they can think of beginning with a given letter. Individuals are given 1 minute to name as many words as possible beginning with each of the letters. The procedure is then repeated for the remaining two letters (Benton, 1994; Strauss, 2006). The test score is the total number of different words produced for all 3 letters.

The COWAT is performed adjacent to the ADAS-Cog₁₁ assessment, i.e., pre-dose at Visit 2 (Baseline/Day 1), and post-dose at approximately 1 hour (±30 minutes) at Visit 3 (Week 2), Visit 4 (Week 6), Visit 5 (Week 12), Visit 7 (Week 20), Visit 8/ET (Week 26), and Visit 9 (Safety follow-up; no dosing). 2. Disease Condition

Alzheimer's Disease Cooperative Study Clinical Global Impression of Change (ADCS-CGIC)

The Alzheimer's Disease Cooperative Study Clinical Global Impression of Change (ADCS-CGIC) scale is a 7-point scale that requires the clinician to assess how much the subject's illness has improved or worsened relative to a baseline state at the beginning of the intervention and rated as: 1, markedly improved; 2, moderately improved; 3, minimally improved; 4, no change; 5, minimally worse; 6, moderately worse; or 7, markedly worse. The ADCS-CGIC consists of 3 parts: a guided baseline interview administered to the subject and caregiver/support person, a follow-up interview administered to the subject and caregiver/support person, and a clinician's rating review (Schneider, 1997). At study start, using the ADCS-CGIC baseline form as a guideline, the ADCS-CGIC rater obtains an integral clinical impression of the subject's status, can apply any formal testing at his/her discretion, and take personal notes regarding the subject's condition; these serve as a reference for future change ratings. The ADCS-CGIC is administered by an experienced clinician who remains independent of the subject's safety, cognitive and functional outcomes, and is trained and certified for this study. The ADCS-CGIC rater ideally remains the same individual for all ADCS-CGIC ratings.

ADCS-CGIC assessments are performed adjacent to ADAS-Cog₁₁ and COWAT assessments pre-dose at Visit 2b (Baseline/Day 1), post-dose at Visit 5 (Week 12), and post-dose at Visit 8/ET (Week 26).

Alzheimer's Disease Cooperative Study—Activities of Daily Living, 23-Item Version (ADCS-ADL23)

The ADCS-ADL23 (Galasko, 1997) is a 23-item assessment of functional impairment in terms of activities of daily living administered to the support person/caregiver. It comprises 23 questions about the subject's involvement and level of performance across items representing daily living. The questions range from basic to instrumental activities of daily living. Each item is rated from the highest level of independent performance to complete loss. The total score range is from 0 to 78, with lower scores indicating greater functional impairment. ADCS-ADL23 assessments are performed pre-dose at Visit 2b (Baseline/Day 1), post-dose at Visit 5 (Week 12), and post-dose at Visit 8/ET (Week 26).

Neuropsychiatric Inventory (NPI)

The NPI is an interview-based rating scale of psychiatric and behavioral disturbances associated with dementia (Cummings, 1994). The support person/caregiver is interviewed by the qualified NPI rater about the presence or absence of 12 symptoms, including delusions, hallucinations, agitation/aggression, depression/dysphoria, anxiety, elation/euphoria, apathy/indifference, disinhibition, irritability/lability, aberrant motor behavior, sleep and nighttime behavior disorders, and appetite and eating disorders. For those symptoms present, the support person/caregiver rates the frequency, severity, and distress of each symptom. A total score and a caregiver distress score are calculated. A higher score indicates more severe psychopathology.

NPI assessments are performed pre-dose at Visit 2b (Baseline/Day 1), post-dose at Visit 5 (Week 12), and post-dose at Visit 8/ET (Week 26).

### 3. Health-Related Quality of Life

EuroQol Group 5-Dimension 5 Level Questionnaire (EQ-5D-5L)

The EQ-5D-5L Health Questionnaire (The EuroQol Group, 1990) is a standardized instrument that offers a simple method for obtaining a self-rating of current health status on a vertical visual analog scale (VAS) of 0-100 (Kind, 1996). Higher scores on the VAS indicate a better health state.

The EQ-5D-5L Health Questionnaire (Proxy version 1) is completed pre-dose at Visit 2b (Baseline/Day 1) and post-dose at Visit 8/ET (Week 26).

### 4. Caregiver Burden/Resource Utilization/Pharmacoeconomic Variables

Zarit Burden Interview (ZBI)

The ZBI is a 22-item questionnaire specifically designed to reflect the stress experienced by caregivers of subjects with dementia (Zarit, 1980). The support person/caregiver answers questions about the impact of the subject's disabilities on their life. The score for each individual item ranges from 0 to 4 (never, rarely, sometimes, quite frequently, and nearly always) with a total score range from 0 to 88; a higher score reflects a higher degree of caregiver perceived burden or stress.

ZBI assessments are performed pre-dose at Visit 2b (Baseline/Day 1) and post-dose at Visit 8/ET (Week 26).

Resource Utilization in Dementia (RUD-Lite®)

The RUD instrument is designed to assess caregiver burden and provide pharmacoeconomic data related to AD (Wimo, 1998). The RUD-Lite®, used in this study, includes baseline and follow-up questions. Caregiver-related questions include a description of the caregiver (demographic information) and time spent caring for the subject and changes in work status. Subject-related questions include residential status and health care resource utilization. Note that the caregiver-related use of health resources included in the RUD is not part of the RUD-Lite®.

RUID-Lite® assessments are performed pre-dose at Visit 2b (Baseline/Day 1) and post-dose at Visit 8/ET (Week 26).

### F. Schedule and Order of Assessments

The study consists of up to 28 days of Screening (Day −28 through Day −6), a Pre-baseline visit (Visit 2a, Day −5 to Day −3), during which subjects undergo an MMSE assessment, and 2 baseline EEG assessments (ERP P300 and qEEG) approximately 2 hours apart, Baseline (Visit 2b, Day 1, randomization), followed by 26 weeks of double-blind treatment, and a 4-week safety follow-up. Note: if 28 days is not sufficient to complete the screening period, the possibility of an extension can be discussed with the Medical Monitor.

For key endpoints, the general order post-dose should be followed:

| | |
|---|---|
| (1) | ADAS-Cog11 |
| (2) | COWAT |
| (3) | EEG assessments (ERP P300 and qEEG) |
| (4) | ADCS-CGIC |

At Pre-baseline visit (Visit 2a, Day −5 to Day −3), MMSE should be done first before all other assessments.

ADAS-Cog₁₁ and COWAT assessments shall occur at clinic visits in the morning at approximately the same time they were performed during the initial Baseline/Day 1 assessment. ADAS-Cog₁₁ and COWAT assessments are performed pre-dose at Baseline/Day 1 (Visit 2b), and post-dose at approximately 1 hour (±30 minutes) at Week 2 (Visit 3), Week 6 (Visit 4) Week 12 (Visit 5), Week 20 (Visit 7), and Week 26 (Visit 8); and additionally at Safety follow up (visit 9; no dosing).

EEG assessments shall occur shortly after the completion of ADAS-Cog₁₁ and COWAT assessments, and before the ADCS-CGIC assessment at applicable visits. EEG assessments are performed at Pre-baseline (Visit 2a), Baseline/Day 1 (Visit 2b), Week 2 (Visit 3), Week 6 (Visit 4), Week 12 (Visit 5), Week 16 (Visit 6), Week 20 (Visit 7), Week 26 (Visit 8), and safety follow up (Visit 9, no dosing).

ADCS-CGIC assessments are organized at adjacent times to the individual EEG assessment times at Baseline/Day 1 (Visit 2), and post-dose at Week 12 (Visit 5), and Week 26 (Visit 8).

PK plasma samples are collected at post-dose at Baseline/Day 1 (Visit 2); pre-dose and post-dose at Week 12 (Visit 5) and Week 26 (Visit 8). The pre-dose PK sample is collected anytime before dosing. The post-dose PK sample is collected anytime between 30 minutes and 120 minutes after dosing as practical. The actual time of dosing and of PK sampling are recorded.

The order of assessments for all other endpoints is flexible.

### G. Statistical Methods

A statistical analysis plan is outlined below.

### Populations for Analysis

### MITT Population

The modified intent-to-treat (mITT) population includes all randomized subjects who took at least one dose of the study medication and who completed at least one ERP P300 baseline assessment and one post-baseline ERP P300 assessment. Subjects are analyzed according to the dose they were randomized to.

### Per Protocol Population

The per protocol population includes all mITT subjects who took the assigned medication during the 26 weeks of treatment, completed at least one ERP P300 plus ADAS-Cog₁₁ and/or COWAT post-baseline assessment, and did not have any major protocol deviations. Subjects are analyzed based on actual treatment received.

### Safety Population

The safety population includes all randomized subjects who received at least one dose of the study medication. Subjects are analyzed based on actual treatment received.

### General Considerations

Descriptive statistics for continuous variables include number of subjects (n), arithmetic mean, standard deviation, median, minimum, maximum and first and third quartile limits unless otherwise noted. Frequency and percentage are calculated for categorical variables.

Change from baseline is calculated by subtracting the baseline score from the observed value at any subsequent visit. For safety summaries, the last pre-randomization measurement is defined as the baseline value. For the primary variable of ERP P300, Pre-baseline and Baseline visit assessments are used for change from baseline comparisons. For other measures, baseline is defined as the last pre-randomization measurement.

Percentages are based on the number of subjects in each treatment group in the given population for AE summary tables, and additionally overall for medical history, prior and concomitant medications. For all other tables, percentages are based on the number of subjects with non-missing data in each treatment group and overall for the given population.

### Analyses

### Primary Analysis—ERP P300 Latency

The primary analysis uses a mixed model for repeated measures (MMRM) to compare the estimated changes between active treatment and placebo in ERP P300 latency. These analyses assess whether or not there is a difference in estimated changes between treatment groups and placebo at Weeks 2, 6, 12, 16, 20, and 26.

### Secondary Analysis

Correlation of ERP P300 with ADAS-Cog11 and COWAT

Secondary analyses include an evaluation of the correlation of ERP P300 latency with cognition/executive memory function, and comparison of treatment and placebo for the Global Statistical Test (GST), ADAS-Cog₁₁, ADCS-CGIC, and ADCS-ADL23.

### Exploratory Analyses

Exploratory endpoints are tested using similar statistical methods described for the primary analysis.

### Subgroup Analyses

Subgroup analyses (e.g., gender, age, MMSE score, AChEI coadministration, ApoE genotype) are performed in the mITT population.

### H. Results

No treatment related Serious Adverse Events were observed in the study. Eight subjects developed treatment related Adverse Events in the placebo treated group, compared to 23 subjects in the 40 mg/day ATH-1017 treated group, and 24 subjects in the 70 mg/day ATH-1017 treated group. The most frequent Adverse Event was injection site reaction, sometimes associated with transient and asymptomatic increases in absolute eosinophil count. Eleven out of 77 subjects terminated early (14%).

The ACT-AD study did not meet the primary endpoint of a statistically significant change in ERP P300 Latency for the modified intent to treat (mITT) population by a mixed model repeated measures (MMRM) analysis when compared with placebo at 26 weeks in a pooled analysis of the 40 mg and 70 mg dose groups. Secondary endpoints, including ADAS-Cog11, ADCS-CGIC, and ADCS-ADL23, were not significant in treated subjects compared with placebo. Surprisingly, however, preliminary results from a post hoc analysis based on the modified intent to treat population suggest ATH-1017 is more effective as a monotherapy than in combination with AChEI therapy. In patients who were not receiving concurrent AChEI therapy, treatment with ATH-1017 stabilized or improved EPR P300 latency (in milliseconds), as shown in Table 1 (decrease in P300 latency from baseline indicates improvement).

TABLE 1
P300 Latency change from baseline (CFB) in
patients receiving ATH-1017 monotherapy

| | | | | | |
|---|---|---|---|---|---|
| W2 | W6 | W12 | W16 | W20 | W26 |

Placebo

| | | | | | | |
|---|---|---|---|---|---|---|
| CFB | 1.7 | 10.7 | 14.9 | 9 | 10.2 | 17.3 |
| (ms) | | | | | | |
| SD | 11.27 | 12.88 | 17.76 | 12.13 | 24.57 | 35.40 |
| N | 8 | 8 | 6 | 6 | 7 | 6 |

ATH-1017

| | | | | | | |
|---|---|---|---|---|---|---|
| CFB | −19.1 | −10.8 | −10.1 | −17.6 | −5.8 | −10.7 |
| (ms) | | | | | | |
| SD | 31.13 | 24.3 | 27.83 | 35.34 | 26.35 | 26.71 |
| N | 20 | 19 | 18 | 15 | 16 | 17 |

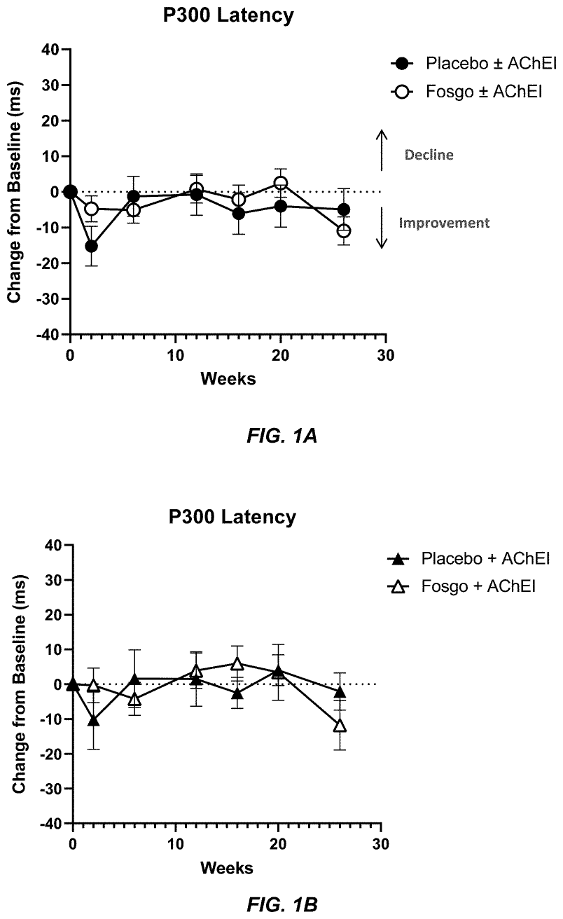

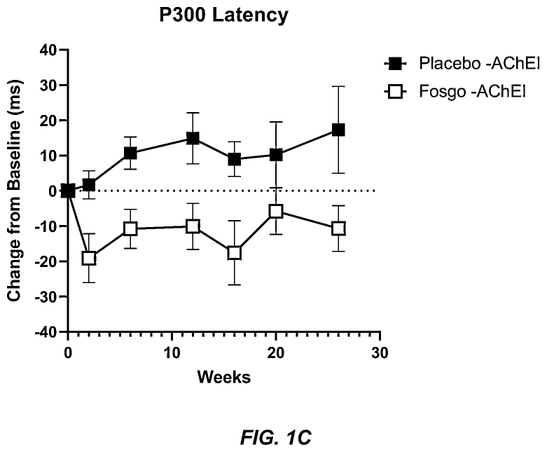

FIG. 1A shows P300 latency change from baseline in the study population, grouped by patients receiving placebo with and without AChEI therapy, and patients receiving fosgonimeton (ATH-1017) with and without AChEI therapy. FIG. 1B and FIG. 1C divide the patients into those on concurrent AChEI therapy and those that are not. The placebo+AChEI group (FIG. 1B) qualitatively stabilized P300 latency compared to placebo alone (FIG. 1C). The fosgonimeton (ATH-1017) alone group also qualitatively stabilized or improved P300 latency compared to placebo (FIG. 1C).

Similarly, treatment with ATH-1017 qualitatively stabilized or improved ADAS-Cog11 scores in patients who were not receiving concurrent AChEI therapy, as shown in Table 2 (increase in ADAS-Cog11 from baseline indicates worsening).

TABLE 2
ADAS-Cog11 change from baseline (CFB) in
patients receiving ATH-1017 monotherapy

| | | | | |
|---|---|---|---|---|
| W2 | W6 | W12 | W20 | W26 |

Placebo

| | | | | | |
|---|---|---|---|---|---|
| CFB | −1.5 | −1 | −0.1 | 3.1 | 4.2 |
| SD | 6.05 | 4.69 | 4.14 | 5.49 | 3.43 |
| N | 8 | 8 | 7 | 7 | 6 |

ATH-1017

| | | | | | |
|---|---|---|---|---|---|
| CFB | −1.4 | −2.1 | −1.5 | −1 | 0.9 |
| SD | 4.6 | 5.45 | 4.03 | 6.3 | 6.74 |
| N | 20 | 20 | 18 | 17 | 18 |

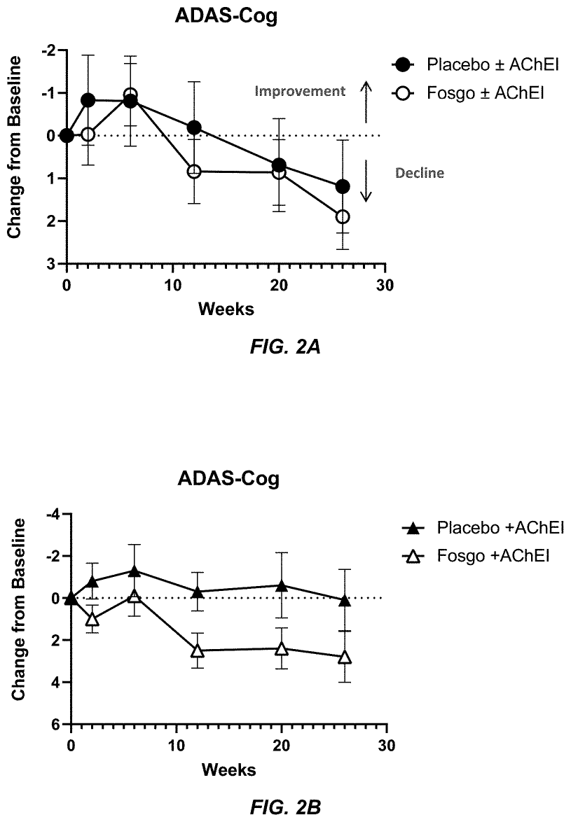

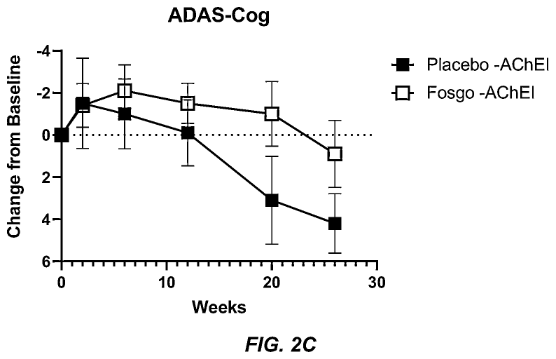

FIG. 2A shows ADAS-Cog score change from baseline in the study population, grouped by patients receiving placebo with and without AChEI therapy, and patients receiving ATH-1017 with and without AChEI therapy. FIG. 2B and FIG. 2C divide the patients into those on concurrent AChEI therapy and those that are not.

In summary, this post hoc analysis based on the modified intent to treat population on fosgonimeton (ATH-1017) monotherapy showed a potentially beneficial change in ERP P300 compared to placebo at 26 weeks (−28 milliseconds) as well as cognitive improvement as measured by ADAS-Cog11 (−3.3 points) compared with placebo at 26 weeks.

ATH-1017 performed similarly as monotherapy with AChEIs in this preliminary analysis, but without the significant adverse events associated with AChEIs. This suggests that ATH-1017 may represent an alternative therapy to AChEIs, sparing the patient he side effects associated with AChEIs, which are associated with a high rate of adverse events and low tolerability.

### Example 3: Open-Label Extension of Studies of Example 1 and Example 2

A. Overview of Study Design

This is a multicenter, open-label extension (OLEX) study of ATH-1017 treatment in subjects with a clinical diagnosis of mild to moderate Alzheimer's disease (AD) who completed 26 weeks treatment in the randomized, placebo-controlled, double-blind studies ATH-1017-AD-0201 (described in Example 1) and ATH-1017-AD-0202 (described in Example 2); hereafter referred to as the ‘parent studies’. The study is conducted at a total of approximately 65 centers across the United States (US) and Australia. Eligible subjects roll over directly from the parent studies, such that Visit 1 (Day 0) of this study is the Week 26 visit from either of the 2 parent studies. All subjects who complete the Week 26 visit of either of the two parent studies and meet the inclusion/exclusion criteria of this Study are assigned to open-label active treatment with ATH-1017 at a dose of 70 mg/day, unless the safety results obtained at Visit 7 of the parent studies suggest otherwise, in which case they are not eligible for this OLEX study. At any time during this OLEX, subjects who do not tolerate open-label study treatment ATH-1017 70 mg/day, and who do not fulfill the stopping criteria, are allowed to have their dose of ATH-1017 adjusted to 40 mg/day; Medical Monitor prior approval is required. Subjects who do not tolerate either ATH-1017 70 mg/day or ATH-1017 40 mg/day, and/or who meet the stopping criteria, are withdrawn from the study.

Study drugs are administered by subcutaneous (SC) injection once-daily (OD), preferably during the daytime. Subjects take their last dose of blinded ATH-1017 (assigned to them in Study ATH-1017-AD-0201 or Study ATH-1017-AD-0202) on site under supervision of site staff at Visit 8/Day 182 of the parent studies (i.e., Visit 1/Day 0 of this OLEX study). The first dose of open-label ATH-1017 from this study is administered by subject or caregiver at the clinic the next day (Visit 2/Day 1), followed by a safety observation window of 1 hour (±15 minutes) post-dose. Subjects must not take more than one dose within 8 hours of the previous dose. The subject should withhold study drug administration on the day of clinic visits; study drug administration is done on site under supervision of site staff at these visits. Each subject is required to have a primary caregiver willing to accept responsibility for supervising or, if required, administering study drug, in accordance with all protocol requirements. During the open-label treatment period, clinic visits take place on Day 0 and thereafter at Day 1, Weeks 2, 6, 12, 18, 22, and 26, and telephone call visits are scheduled for Week 1 (Day 7), and Week 4 (Day 28). A safety follow-up visit is scheduled 2 weeks after completion of the open-label period at Week 28. Subjects may live at home, in a senior residential setting, or an institutional setting without the need for continuous nursing care. The end of the study is defined as the date of the safety follow-up visit, Visit 11/Week 28. Subjects who terminate prior to Visit 10 are to complete same assessments as Visit 10/early termination (ET) within 2 weeks of the date of ET.

An independent Data Safety Monitoring Board conducts periodic review and assessments of safety data (AEs, labs, ECG, etc.) throughout the study to ensure the safety of study subjects.

### B. Subject Population

Subjects are not randomized to treatment in this OLEX study. All subjects who complete the Week 26 visit of either of the 2 parent studies, and meet the inclusion/exclusion criteria of Study ATH-1017-AD-0203, with no safety concerns from results obtained at Visit 7 of either of the 2 parent studies, are assigned to open-label active treatment with ATH-1017 at a dose of 70 mg/day. Subjects who do not tolerate ATH-1017 70 mg/day during this study are allowed to have their dose adjusted to 40 mg/day; Medical Monitor prior approval is required.

It is estimated that up to approximately 300 subjects will enter this OLEX study; subjects will roll over from Studies ATH-1017-0201 and ATH-1017-AD-0202 (the parent studies).

All subjects must meet all the inclusion criteria and none of the exclusion criteria.

Protocol exemptions related to enrollment criteria are only allowed with prior Investigator and Sponsor approval, supported by documented agreement from the IRB/IEC.

Evaluation of safety lab data from Visit 7 of either of the 2 parent studies is allowed for assessment of subject eligibility. Subjects who are considered by the investigator to have safety or tolerability issues at Visit 7 of either of the 2 parent studies should be carefully re-considered for transition into this OLEX study at the final study visit of Study ATH-1017-AD-0201 or Study ATH-1017-AD-0202 (i.e., Visit 1 of this study, ATH-1017-AD-0203). Safety lab data from Visit 7 of the initial studies is to be confirmed with safety lab data from Visit 1 of this study. Subjects who are subsequently found to be not eligible based on safety lab data from Visit 1 are discontinued.

### Inclusion Criteria

1) Subject has completed the Week 26 visit of either of the 2 parent studies.
2) Male subjects and their partners must agree to continue to use a double-barrier method of contraception during the study, including the follow-up period, unless the partner is not of childbearing potential. Only female subjects of non-childbearing potential (i.e., permanently sterilized, postmenopausal) are eligible for participation.
3) Reliable and capable support person/caregiver, who is willing to accept responsibility for supervising the treatment or, if required, administering study drug in accordance with all protocol requirements. The support person/caregiver must see the subject at least once-daily for administering or supervising dose administration.
4) Subject capable of giving signed informed consent, which includes compliance with the requirements and restrictions listed in the Informed Consent Form and in this protocol. If the subject is incapable of giving informed consent in the judgment of the investigator then consent may be provided by a legally acceptable representative.
5) Written informed consent from a) the subject or legally acceptable representative and b) caregiver/support person has been obtained prior to any study-related procedures, including prior to initiating procedures to evaluate eligibility for the study.
6) Written documentation has been obtained in accordance with the relevant country and local privacy requirements, where applicable (e.g., Written Authorization for Use and Release of Health and Research Study Information).
7) Subjects and caregivers/support persons are able, as assessed by the investigator, and willing to follow study instructions and likely to complete all required study visits.
8) Subjects must be in generally good health as assessed by the investigator from medical history and physical/neurological examination, vital signs, ECG, and standard laboratory tests.

### Exclusion Criteria

1) Subject has experienced a serious treatment-related AE during either of the 2 parent studies, which in the opinion of the investigator could present an increased safety risk to the subject during the OLEX study, in discussion with the Medical Monitor.
2) New diagnosis of severe major depressive disorder even without psychotic features. Any subject with formalized delusions or hallucinations are excluded.
3) Significant suicide risk as defined by suicidal ideation based on the C-SSRS at Visit 1 (i.e., a ‘yes’ response to Question 4 or 5, or any specific behaviours).
4) Newly-diagnosed malignant tumor, except for the following conditions that are stable in the judgement of the investigator:
   a) Adequately treated squamous and basal cell carcinoma, or squamous and basal cell carcinoma in situ
   b) Prostate carcinoma in situ
5) The subject has a condition or is in a situation which, in the investigator's opinion, may put the subject at significant risk, may confound the study results, or may interfere significantly with the subject's compliance or participation in the study.

### C. Drug Product

Pre-filled syringes of ATH-1017 at 40 mg contain 1.0 mL of 40 mg/mL ATH-1017 in a solution of 10 mM sodium phosphate and 0.5% NaCl. Pre-filled syringes of ATH-1017 at 70 mg contain 1.0 mL of 70 mg/mL ATH-1017 in a solution of 10 mM sodium phosphate. Each pre-filled syringe of placebo contains 1.0 mL of a solution of 10 mM sodium phosphate and 1.1% NaCl. All solutions are adjusted to pH of approximately 7.6.

ATH-1017 and Placebo are Administered Subcutaneously.

### D. Dose Modification

In this OLEX study, treatment allocation is not blinded; all subjects receive open-label ATH-1017 at a dose of 70 mg/day. An option to reduce the dose of ATH-1017 to 40 mg/day is available.

### E. Endpoints

The objective of this study is the extended determination of the safety and tolerability of ATH-1017 in subjects with mild to moderate AD who completed the 26-week randomized treatment in Study ATH-1017-AD-0201 or Study ATH-1017-AD-0202.

Safety and tolerability are assessed by analysis of adverse events (AEs), including injection site AEs; changes from baseline for the following variables: vital signs, 12-lead electrocardiogram (ECG), and laboratory tests (chemistry, hematology, urinalysis); concomitant medication assessments, physical and neurological exams, and Columbia-Suicide Severity Rating Scale (C-SSRS).

### F. Schedule and Order of Assessments

Following informed consent and confirmation of eligibility at Visit 1, the study consists of 26 weeks of open-label treatment and a 2-week safety follow-up.

### G. Statistical Methods

A statistical analysis plan (SAP) is issued.

### H. Results

Treatment herein with ATH-1017 achieves one or more of the primary, secondary, or exploratory endpoints, while having acceptable safety and tolerability.

Unless otherwise defined, all technical and scientific terms used herein have the same meaning as commonly understood by one of ordinary skill in the art to which this disclosure belongs.

Thus, it should be understood that although the present disclosure has been specifically disclosed by preferred embodiments and optional features, modification, improvement and variation of the disclosures embodied therein herein disclosed may be resorted to by those skilled in the art, and that such modifications, improvements and variations are considered to be within the scope of this disclosure. The materials, methods, and examples provided here are representative of preferred embodiments, are exemplary, and are not intended as limitations on the scope of the disclosure.

It is to be understood that while the disclosure has been described in conjunction with the above embodiments, that the foregoing description and examples are intended to illustrate and not limit the scope of the disclosure. Other aspects, advantages and modifications within the scope of the disclosure are apparent to those skilled in the art to which the disclosure pertains.

### REFERENCES

Aisen, P. S., et al. (2008). High-dose B vitamin supplementation and cognitive decline in Alzheimer disease: a randomized controlled trial. JAMA 300(15), 1774-1783.
Aisen, P. S., et al. (2003). Effects of rofecoxib or naproxen vs placebo on Alzheimer disease progression: a randomized controlled trial. JAMA 289(21), 2819-2826.
Ally et al. (2006) The P300 component in patients with Alzheimer's disease and their biological children. Biopsychology 72, 180-187. Cecchi, M., et al. (2015). A clinical trial to validate event-related potential markers of Alzheimer's disease in outpatient settings. Alzheimer's & Dementia: Diagnosis, Assessment, & Disease Monitoring 1, 387-394. PMID 27239520.
Aricept (donepezil hydrochloride) tablets prescribing information. Revised: July 2015. © 2012 Eisai Inc.
Benton, A. L., Hamsher, K., Rey, G. L., & Sivan, A. B. (1994). Multilingual Aphasia Examination (3rd ed.). Iowa City, I A: AJA Associates.
Citron, M. (2010). Alzheimer's disease: strategies for disease modification. Nature Reviews Drug Discovery 9, 387-398.
Cummings J L, Mega M, Gray K, Rosenberg-Thompson S, Carusi D A, Gornbein J. The Neuropsychiatric Inventory: comprehensive assessment of psychopathology in dementia. Neurology 1994; 44: 2308-2314.
Ebens, A., et al. (1996). Hepatocyte growth factor/scatter factor is an axonal chemoattractant and a neurotrophic factor for spinal motor neurons. Neuron 17:1157-1172.
EuroQol Group (1990). “EuroQol—a new facility for the measurement of health-related quality of life”. Health Policy (Amsterdam, Netherlands). 16 (3):199-208.
FDA Guidance for Industry, Drug-Induced Liver Injury: Premarketing Clinical Evaluation, July 2009.
Folstein M F, Folstein S E, McHugh P R. (1975). “Mini-mental state”. A practical method for grading the cognitive state of patients for the clinician. J Psychiatr Res 12(3), 189-198.
Funakoshi, H., and Nakamura, T. (2011). Hepatocyte Growth Factor (HGF): Neurotrophic functions and therapeutic implications for neuronal injury/diseases. Current Signal Transduction Therapy 6:156-167.
Galasko D, Bennett D, Sano M, Ernesto C, Thomas R, Grundman M, et al. (1997). An inventory to assess activities of daily living for clinical trials in Alzheimer's disease. The Alzheimer's disease Cooperative Study. Alzheimer's Disease and Associated Disorders, 11(S2), S33-S39.
Hamasaki, H., et al. (2014). Down-regulation of MET in hippocampal neurons of Alzheimer's disease brains. Neuropathology 34, 284-290. PMID 24444253.
Hawrylycz, M., et al. (2015). Canonical genetic signatures of the adult human brain. Nature Neuroscience 18, 1832-1844. PMID 26571460. Hyman, B. T., et al. (2012). National Institute on Aging—Alzheimer's Association guidelines on neuropathologic assessment of Alzheimer's disease. Alzheimer's & Dementia: The Journal of the Alzheimer's Association 8, 1-13.
Hilt D C, Dgetluck N, Sainati S, Moebius H J. Effect of circadian rhythm on cognition testing in a phase 2B mild/moderate Alzheimer's Disease (A D) study. J Prev Alzheimers Dis. 2015:3(4): 335.
Hughes C P, Berg L, Danziger W L, Coben L A, Martin R L. (1982). A new clinical scale for the staging of dementia. Br J Psychiatry 140, 566-572.
Kind P. The EuroQoL instrument: an index of health-related quality of life. In: Spilker B, ed. Quality of Life and Pharmacoeconomics in Clinical Trials, 2nd edn. Philadelphia, Pa.: Lippincott-Raven Publishers, 1996:191-201.
Leiser, S. C., Dunlop, J., Bowlby, M. R., Devilbiss, D. M. (2011). Aligning strategies for using EEG as a surrogate biomarker: a review of preclinical and clinical research. Biochemical Pharmacology 81:1408-1421.
Maina, F., and Klein, R. (1999). Hepatocyte growth factor, a versatile signal for developing neurons. Nature Neuroscience 2(3):213-217.
McKhann G M, Knopman D S, Chertkow H, Hyman B T, Jack Jr C R, Kawas C H, et al. The diagnosis of dementia due to Alzheimer's disease: recommendations from the National Institute of Aging-Alzheimer's Association workgroups on diagnostic guidelines for Alzheimer's disease. Alzheimers Dement. 2011; 7:263-9.
Molnarfi, N., Benkhoucha, M., Funakoshi, H., Nakamura, T., and Lalive, P. H. (2015). Hepatocyte growth factor: A regulator of inflammation and autoimmunity. Autoimmun Rev 14, 293-303.
O'Brien, P. C. (1984). Procedures for comparing samples with multiple endpoints. Biometrics 40, 1079-1087.
Olichney et al. (2011) Cognitive Event-Related Potentials: Biomarkers of Synaptic Dysfunction Across the Stages of Alzheimer's Disease. Journal of Alzheimer's Disease 26:215-228.
Organ, S. L, Tsao, M-S. (2011). An overview of the c-MET signaling pathway. Ther Adv Med Oncol 3(1):S7-S19.
Piguet et al. (2017) Clinical Gene Therapy for Neurodegenerative Diseases: Past, Present, and Future. Human Gene Therapy 28(11):988-1003.
Rosen W G, Mohs R C, Davis K L (1984). A new rating Scale for Alzheimer's disease. Am J Psychiatry 141:1356-1364.
Sano, M., et al. (1997). A controlled trial of selegiline, alpha-tocopherol, or both as treatment for Alzheimer's disease. New England Journal of Medicine 336(17), 1216-1222.
Schneider L S, Olin J T, Doody R S, Clark C M, Morris J C, Resiberg B, et al. Validity and reliability of the Alzheimer's Disease Cooperative Study—Clinical Global Impression of Change. The Alzheimer's Disease Cooperative Study. Alzheimer Dis Assoc Disord. 1997; 11(2):S22-S32.
Shang, J., et al. (2011). Strong neurogenesis, angiogenesis, synaptogenesis, and antifibrosis of hepatocyte growth factor in rats brain after transient middle cerebral artery occlusion. J. Neurosci. Res. 89, 86-95.
Sheikh J I, Yesavage J A. (1986). Geriatric Depression Scale (GDS): Recent evidence and development of a shorter version. Clinical Gerontologist: The Journal of Aging and Mental Health, 5(1-2), 165-173.
Strauss, E., Sherman, E. M. S., & Spreen, O. (2006). Compendium of neuropsychological tests: Administration, norms, and commentary (3rd ed., p. 502). Oxford: Oxford University Press.
Tyndall, S. J et al. (2007). Hepatocyte growth factor-induced enhancement of dendritic branching is blocked by inhibitors of N-methyl-D-aspartate receptors and calcium/calmodulin-dependent kinases. J. Neurosci. Res. 85, 2343-2351.
Wimo A, Wetterholm A L, Mastey V, Winblad B. Evaluation of the resource utilization and caregiver time in Anti-dementia drug trials—a quantitative battery. In: Wimo A, Karlsson G, Jonsson B, Winblad B (eds). The Health Economics of Dementia, 1998. Wiley's, London, U K.
Zarit, S H, Reever, K E, Back-Peterson, J. Relatives of the impaired elderly: correlates of feelings of burden. The Gerontologist. 1980; 20:649-655.

1. A method of slowing the decline in cognition or improving cognition in a patient diagnosed with mild to moderate Alzheimer's Disease (AD), comprising administering to a patient in need thereof 2-90 mg per day of a compound of formula A19 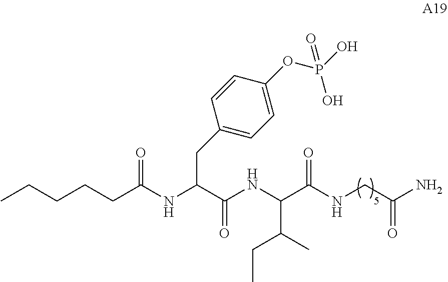
or a pharmaceutically acceptable salt thereof,
wherein the slowing of the decline or the improvement is determined after administering the treatment for at least 6 weeks.

2-5. (canceled)

6. The method of claim 1, wherein the patient is acetylcholinesterase inhibitor (AChEI) naïve.

7. The method of claim 1, wherein the patient received an AChEI in the past and preferably discontinued AChEI therapy at least 4 weeks prior to administration of the compound.

8. The method of claim 1, which reduces the rate of decline, stabilizes, or improves ADAS-Cog11.

9. (canceled)

10. The method of claim 1, wherein the patient has a Mini-Mental State Examination (MMSE) score of at least 14, between 14 and 24, between 15 and 24, between 16 and 24, between 17 and 24, between 18 and 24, between 19 and 24, between 20 and 24, between 21 and 24, between 22 and 24, between 23 and 24 prior to the start of treatment with the compound of formula A19.

11-14. (canceled)

15. The method of claim 1, wherein the patient has a Clinical Dementia Rating (CDR) Scale global score of 1 or 2 prior to the start of treatment with the compound of formula A19.

16. The method of claim 1, wherein the patient is between age 55 and 85.

17. The method of claim 1, wherein the compound of formula A19 or the pharmaceutically acceptable salt thereof is administered by subcutaneous injection.

18. (canceled)

19. The method of claim 1, comprising administering the compound of formula A19 or the pharmaceutically acceptable salt thereof at a dose of 40 mg or 70 mg.

20-21. (canceled)

22. The method of claim 1, comprising administering the compound of formula A19 or the pharmaceutically acceptable salt thereof for 26 weeks or more.

23-25. (canceled)

26. The method of claim 1, which slows the decline or improves in the ability to perform basic to instrumental activities of daily living and verbal fluency in the patient.

27-28. (canceled)

29. The method of claim 1, wherein cognitive capacity is assessed by determining the patient's score before and after administration of the compound of formula A19 or the pharmaceutically acceptable salt thereof using an 11-item Alzheimer's Disease Assessment Scale—cognitive subscale (ADAS-Cog11).

30-33. (canceled)

34. The method of claim 29, wherein the effect on ADAS-Cog11 and/or ADCS CGIC is maintained for at least 2 weeks or at least 4 weeks after the end of treatment.

35. The method of claim 1, which reduces the rate of decline, stabilizes, or improves at least one, at least two, at least three, at least four, at least five, at least six, at least seven, at least eight, at least nine, or at least ten subdomains of a Neuropsychiatric inventory (NPI).

36. The method of claim 1, which reduces the rate of decline, stabilizes, or improves a NPI score, Alzheimer's disease cooperative study-activities of daily living, 23-item version (ADCS-ADL23) score and/or Controlled Oral Word Association Test (COWAT) score.

37-38. (canceled)

39. The method of claim 1, which reduces the rate of decline, stabilizes, or improves a Resource utilization in dementia lite version (RUD-lite) scale, a EQ-5D-5L score, and/or Zarit burden interview (ZBI) score.

40-49. (canceled)

50. The method of claim 1, which improves serum NFL levels.

51. The method of claim 1, which improves serum NFL levels, and at least one of phospho-tau, ABeta 1-42, and/or YLK41 levels.

52-54. (canceled)

55. The method of claim 1, comprising administering a monosodium salt of the compound of formula A19.

56. (canceled)
